(19) United States
(12) Patent Application Publication
(10) Pub. No.: US 2025/0311918 A1
(43) Pub. Date: Oct. 9, 2025

(54) MANUFACTURING METHOD OF BENDING TUBE, BENDING TUBE, AND INSERTION INSTRUMENT

(71) Applicant: OLYMPUS MEDICAL SYSTEMS CORP., Tokyo (JP)

(72) Inventors: Koichi TSURUTA, Yokohama-shi (JP); Kohki KAMATA, Tokyo (JP)

(73) Assignee: OLYMPUS MEDICAL SYSTEMS CORP., Tokyo (JP)

(21) Appl. No.: 19/097,064

(22) Filed: Apr. 1, 2025

Related U.S. Application Data
(60) Provisional application No. 63/631,469, filed on Apr. 9, 2024.

(51) Int. Cl. A61B 1/008; A61B 1/00; B23K 26/22; B23K 26/28

(52) U.S. Cl. CPC A61B 1/008; A61B 1/0011; B23K 26/22; B23K 26/28

(57) ABSTRACT

A method of manufacturing a bending tube for use with an insertion instrument according to one aspect of the present disclosure includes: forming a first joint ring from a sheet material; forming a second joint ring from the sheet material; forming an abutting portion in the second joint ring, wherein the abutting portion is configured to abut against an end surface of the first joint ring, and wherein the abutting portion extends in a direction intersecting a longitudinal axis; and joining the abutting portion to the end surface of the first joint ring.

### RELATED APPLICATION DATA

This application is based on and claims priority under 35 U.S.C. § 119 to U.S. Provisional Application No. 63/631,469 filed on Apr. 9, 2024, the entire contents of which are incorporated herein by reference.

### FIELD OF DISCLOSURE

The present disclosure relates to a manufacturing method of a bending tube provided in a bending portion of an insertion instrument such as an endoscope, and to a bending tube, and an insertion instrument.

### BACKGROUND

In general, an endoscope which is an insertion instrument includes an elongated insertion portion configured to be inserted into a body cavity. The insertion portion is connected to an operation portion located on a proximal end side. The insertion portion includes a distal end portion, a bending portion, and a flexible tube portion. The distal end portion is constituted of a distal end rigid portion (distal-end constituting member). The distal end portion is connected to the distal end of the bending portion. The bending portion is connected to the distal end of the flexible tube portion. The bending portion is operated to be bent to vary the direction of the distal end portion. The flexible tube portion is a tubular body which is elongated and has flexibility.

The bending portion of the insertion portion includes a bending tube, a braid configured of a metal braided tube and the like and covering the bending tube, and an outer cover that covers the exterior side of the braid. The outer cover is a tubular body such as a rubber member that covers the outer circumferential surface of the bending tube. The bending tube includes a plurality of joint rings. The plurality of joint rings are disposed side by side in the longitudinal direction of the bending portion. The adjacent joint rings are rotatably coupled with each other.

A configuration, a manufacturing method, and the like of a bending tube provided in such a bending portion are disclosed, for example, in Japanese Patent Application Laid-Open Publication Nos. 2010-252859 and 2007-159636, or WO No. 2016/190011.

Japanese Patent Application Laid-Open Publication Nos. 2010-252859 and 2007-159636 disclose a technology for manufacturing a bending tube to be used in an endoscope by pressing. In addition, WO No. 2016/190011 discloses a configuration for press-working a part of a shape of a bending tube to be used in an endoscope, and welding a bending operation wire or a bending operation wire mounting tool to joint rings of the bending tube.

### SUMMARY

A method of manufacturing a bending tube for use with an insertion instrument according to one aspect of the present disclosure includes: forming a first joint ring from a sheet material; forming a second joint ring from the sheet material; forming an abutting portion in the second joint ring, wherein the abutting portion is configured to abut against an end surface of the first joint ring. The abutting portion extends in a direction intersecting a longitudinal axis; and joining the abutting portion to the end surface of the first joint ring.

An insertion instrument according to one aspect of the present disclosure includes: an insertion portion configured to be inserted into a subject; and a bending tube that is mounted to a distal end side of the insertion portion and includes a plurality of joint rings. The plurality of joint rings include: first joint rings having a first diameter centered about a longitudinal axis, the first joint rings being formed by press-working a sheet material made of a metal plate; second joint rings having a second diameter centered about the longitudinal axis, the second diameter being different from the first diameter, the second joint rings being formed by press-working the sheet material; at least one abutting portion provided integrally with at least one of the first joint rings or the second joint rings by bending the at least one of the first joint rings or the second joint rings, and configured to abut against a surface of an end portion in a direction of the longitudinal axis of the other of the first joint rings or the second joint rings; and a joining portion that joins the abutting portion and the surface of the end portion in the direction of the longitudinal axis.

### BRIEF DESCRIPTION OF THE DRAWINGS

FIG. 1 is a view showing a configuration of an endoscope.

FIG. 2 is a cross-sectional view showing a configuration of a distal end portion and a bending portion of an insertion portion.

FIG. 3 is a plan view showing a working sheet that has gone through a plurality of processing stages.

FIG. 4 is a plan view showing a first press working sheet.

FIG. 5 is a plan view showing a second press working sheet.

FIG. 6 is a plan view showing a bending tube processing sheet.

FIG. 7 is a cross-sectional view showing a pressing process for causing a burring processed portion to expand by using a press jig.

FIG. 8 is a cross-sectional view showing a joint portion.

FIG. 9 is a plan view showing the bending tube processing sheet, with both side portions of respective joint ring preparatory bodies separated from a carrier.

FIG. 10 is a view showing a state where each of the joint rings has been subjected to bending processing.

FIG. 11 is a plan view showing the bending tube processing sheet in the state where each of the joint rings has been subjected to the bending processing.

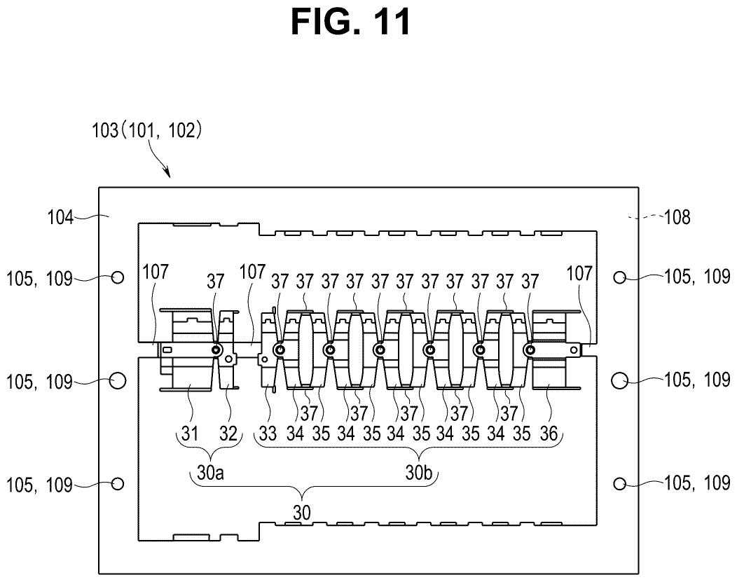

FIG. 12 is a view showing a state where a fitted part of a first joining end portion and a second joining end portion of each of the joint rings subjected to the bending processing is spot-welded.

FIG. 13 is a view showing a state before a first joint ring group and a second joint ring group are joined.

FIG. 14 is a view showing a state where the first joint ring group and the second joint ring group are joined.

FIG. 15 is a perspective view partly showing a state where a first joint-welding portion and a second joint-welding portion are fitted to each other.

FIG. 16 is a partial cross-sectional view showing a state where the first joint-welding portion and a second joint-welding portion are fitted to each other, to be laser-welded.

FIG. 17 is a partial cross-sectional view showing a dimensional relationship between the first joint-welding portion and the second joint-welding portion.

FIG. 18 is a plan view partially showing the dimensional relationship between the first joint-welding portion and the second joint-welding portion.

FIG. 19 is a partial cross-sectional view showing a joining part of the first joint ring group and the second joint ring group.

FIG. 20 is a partial cross-sectional view showing a state where an end surface on a proximal end side of the second joint-welding portion contacts an end surface on a distal end side of a first middle joint ring, when the bending portion is maximally bent.

FIG. 21 relates to a first modified example and is a view showing a form in which four bending operation wires are fixed to a distal-most joint ring.

FIG. 22 relates to the first modified example and is a view showing a form in which two bending operation wires are fixed to the distal-most joint ring.

FIG. 23 relates to a second modified example and is a perspective view partially showing a state where a joining form of the first joint-welding portion and the second joint-welding portion is viewed from a distal end upper side.

FIG. 24 relates to the second modified example and is a perspective view partially showing a state where the joining form of the first joint-welding portion and the second joint-welding portion is viewed from a proximal end upper side.

FIG. 25 relates to the second modified example and is a perspective view partially showing a state where another joining form of the first joint-welding portion and the second joint-welding portion, which is different from the one shown in FIG. 23 and FIG. 24, is viewed from the distal end upper side.

FIG. 26 relates to the second modified example and is a perspective view partially showing a state where the other joining form of the first joint-welding portion and the second joint-welding portion, which is different from the one shown in FIG. 23 and FIG. 24, is viewed from the proximal end upper side.

FIG. 27 relates to the second modified example and is a perspective view partially showing a state where another joining form of the first joint-welding portion and the second joint-welding portion, which is different from the one shown in FIG. 26, is viewed from the proximal end upper side.

FIG. 28 relates to a first reference example, and is a cross-sectional view showing a form in which stepped pins that join left and right sides of a first linking joint ring and a second linking joint ring are provided.

FIG. 29 relates to the first reference example, and is a cross-sectional view showing a form in which pins that join the left and right sides of the first linking joint ring and the second linking joint ring are provided.

FIG. 30 relates to a second reference example, and is a perspective view partially showing a state where a recessed portion, which is formed at an upper portion of a first joint-welding portion, is viewed from a distal end upper side.

FIG. 31 relates to the second reference example, and is a partial cross-sectional view showing a form in which a first linking joint ring and a second joining linking ring are surface-joined by recessed portions formed respectively on upper and lower portions of the first linking joint ring and the second linking joint ring.

FIG. 32 relates to the second reference example, and is a perspective view partially showing a state where a recessed portion formed on a side portion of the first joint-welding portion is viewed from the distal end upper side.

FIG. 33 relates to the second reference example, and is a partial cross-sectional view showing a form in which the first linking joint ring and the second linking joint ring are surface-joined by recessed portions formed respectively on left and right portions of the first linking joint ring and the second linking joint ring.

FIG. 34 relates to a third reference example, and is a partial cross-sectional view showing a bending tube in a state where wire guides are formed integrally with joint portions and bent at a predetermined angle.

FIG. 35 relates to the third reference example, and is a partial cross-sectional view showing a configuration of the wire guide formed integrally with the joint portion.

FIG. 36 relates to the third reference example, and is a partial cross-sectional view showing a configuration of curvatures of an inner circumferential surface on a proximal end side of the wire guide.

FIG. 37 relates to the third reference example, and is a partial cross-sectional view showing a configuration of curvatures of the inner circumferential surface on a distal end side and the proximal end side of the wire guide.

FIG. 38 relates to a fourth reference example, and is a view showing a configuration of a recessed portion that is a deformed part of a hole portion of a protrusion portion of each of the joint rings constituting a joint portion.

### DETAILED DESCRIPTION

Incidentally, a bending tube is configured by layering sheet materials subjected to press working similarly as in the conventional technique and joining a plurality of adjacent joint rings having the same diameter to each other. However, when the bending tube is fabricated using the press-worked sheet materials, it is difficult to join two joint rings having different diameters integrally.

Therefore, in conventional techniques or a combination thereof, there is a problem of a high cost when a bending tube including joint rings having a diameter change, is manufactured. In other words, the bending tube fabricated using the press-worked sheet materials has restrictions on forming in terms of the change in the diameters of the joint rings. Therefore, the bending tube fabricated using the press-worked sheet materials has had a problem that the bending tube cannot have a diameter change such as a desirable diameter expansion structure.

Thus, in the bending tube fabricated by conventional press working, there has been a problem of a difficulty in forming the bending portion having the diameter change for eliminating an increase in a burden on a patient, and influences on an insertion performance into a subject, an observation performance, and the like.

The present disclosure has been achieved in view of the above-described circumstances, and an object of the present disclosure is to provide a manufacturing method of a bending tube that can be fabricated at a low cost, the bending tube, and an insertion instrument by achieving a reduced cost by simplifying the manufacturing method of the bending tube for an endoscope having a diameter change. Accordingly, the bending tube in the present disclosure is used in a bending portion of an insertion portion of an insertion instrument such as an endoscope, to thereby achieve a configuration that eliminates an increase in a burden on a patient, and influences on an insertion performance into a subject, an observation performance, and the like.

### Embodiments

Hereinafter, description will be made by taking an endoscope, as an insertion instrument in the present embodiment, as an example. Note that, in the description below, the drawings based on each embodiment are schematic. In addition, care should be taken to the fact that, in the respective constituent elements of the endoscope, the relationship between thicknesses and widths of respective parts, a ratio of a thickness of a certain part to that of another part, and the like in the drawings are different from the actual ones. Furthermore, in the respective constituent elements of the endoscope, the respective drawings sometimes include parts in which the relationships and ratios among the dimensions of the elements are different.

First, description will be made on a schematic configuration of an endoscope 1, which is an insertion instrument. Note that the endoscope 1 in the present embodiment has a configuration applicable to various endoscopes such as an upper gastrointestinal endoscope, a lower gastrointestinal endoscope, a bronchoscope, an ureteropelvic endoscope, a duodenoscope, and the like.

In addition, the endoscope 1, which is the insertion instrument, may be a single-use type which is to be used once and configured such that all or at least a part of elements constituting the endoscope will be disposed of after use, or may be a reuse type to be reprocessed after use and reused.

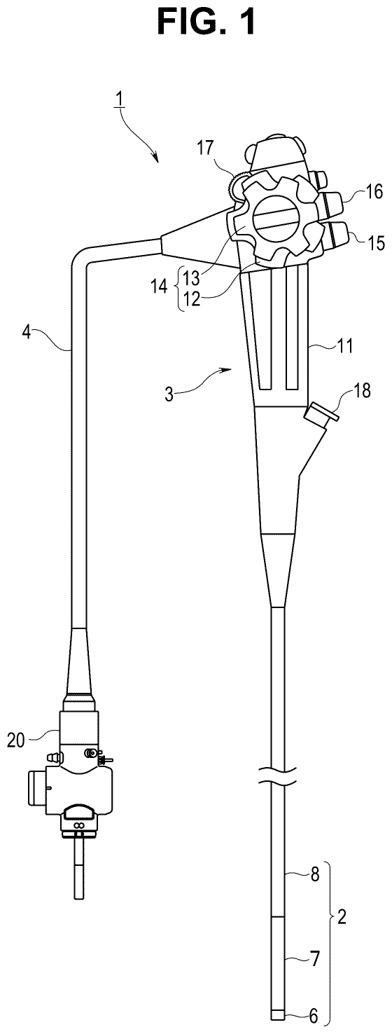

The endoscope 1 in the present embodiment shown in FIG. 1 is an insertion instrument including an insertion portion 2, an operation portion 3, and a universal cable 4. The insertion portion 2 is a long and thin member configured to be inserted into a subject. The universal cable 4 is a composite cable. The insertion portion 2 includes, in the following order from the distal end, a distal end portion 6, a bending portion 7, and a flexible tube portion 8.

The operation portion 3 includes a bending operation knob 14 which is rotatably disposed and configured to operate and bend the bending portion 7 of the insertion portion 2. In addition, the operation portion 3 includes a gas/liquid feeding button 15, a suction button 16, a treatment instrument raising base (forceps elevator) operation lever 17, and the like.

The bending operation knob 14 includes two substantially disk-shaped rotation knobs that are superimposed one above the other. The two rotation knobs include a UD bending operation knob 12 and an RL bending operation knob 13. The UD bending operation knob 12 is an operation member for operating to bend the bending portion 7 in up and down directions. The RL bending operation knob 13 is an operation member for operating to bend the bending portion 7 in left and right directions.

The operation portion 3 includes a grasping portion 11 and a treatment instrument insertion channel insertion portion 18. The grasping portion 11 is a part to be grasped by a user.

The treatment instrument insertion channel insertion portion 18 is disposed at the grasping portion 11. The treatment instrument insertion channel insertion portion 18 is an opening portion through which various kinds of treatment instruments are inserted into the treatment insertion channel disposed in the insertion portion 2. The treatment instrument insertion channel insertion portion 18 includes a forceps plug.

The universal cable 4 is extended from the operation portion 3. The universal cable 4 includes, at an extension end thereof, an endoscope connector 20 configured to be attachable to and detachable from a light source apparatus, not shown. Although not shown here, a coil-shaped coil cable is connected to the endoscope connector 20, and at an extension end of the coil cable, an electric connector, which is attachable to and detachable from a video processor (not shown), is provided. Note that, as the shape of the endoscope connector 20, various shapes can be considered as needed, other than the shape shown in the drawing.

Note that the endoscope 1 in the present embodiment is configured such that illumination light is transmitted from the light source apparatus (not shown) to the distal end portion 6 with a light guide bundle (not shown), which is an illumination means, inserted through the insertion portion 2, the operation portion 3, and the universal cable 4.

Next, detailed description will be made below on the bending portion 7, which is a tubular portion that constitutes a part of the insertion portion 2 of the endoscope 1. The bending portion 7 includes, at a part to be bent, a bending part which is a snake bone part.

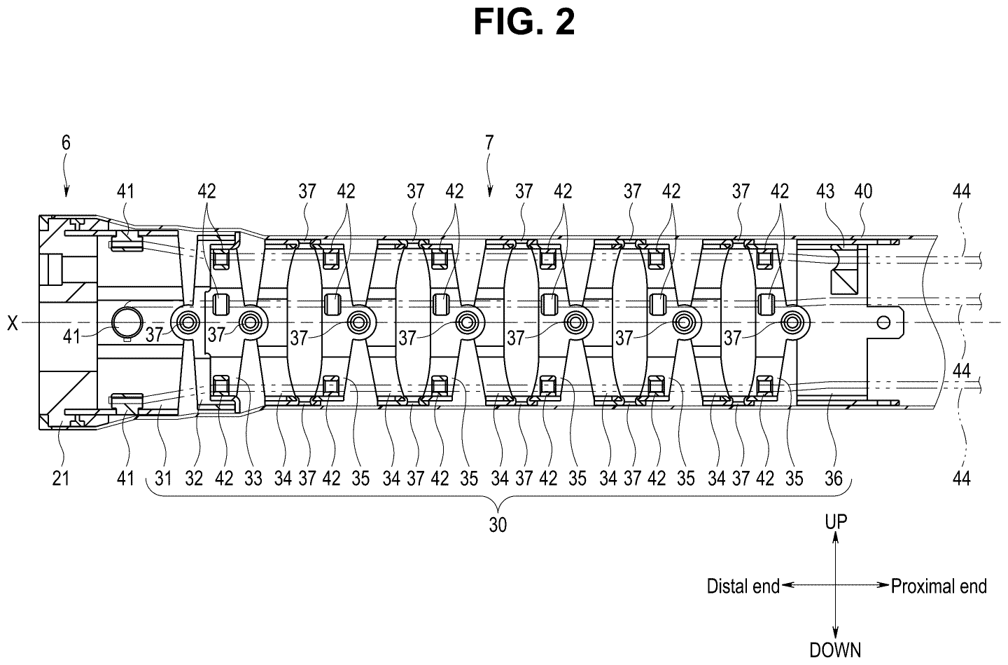

As shown in FIG. 2, the insertion portion 2 includes a distal-end constituting portion 21, which is a substantially cylindrical-shaped distal end rigid member incorporated in the distal end portion 6. To the distal-end constituting portion 21, a distal end of a bending tube 30 constituted of a plurality of joint rings is connected. The bending tube 30 is constituted of a plurality of joint rings 31, 32, 33, 34, 35, and 36, which are bending pieces having a substantially annular shape and connected to each other along a longitudinal axis X as the center axis of the insertion portion 2 when the insertion portion 2 is in a linear state.

Specifically, the bending tube 30 includes, in the following order from the distal end side, the distal-most joint ring 31, the first linking joint ring 32 (first joint ring), and the second linking joint ring 33 (second joint ring) that are coupled with each other along the longitudinal axis X, the plurality of first middle joint rings 34 (third joint ring) and the plurality of second middle joint rings 35 that are alternately coupled with each other, and the proximal-most joint ring 36.

The plurality of joint rings 31, 32, 33, 34, 35, and 36 are rotatably coupled with each other by joint portions 37 which are pivotal parts. The distal-most joint ring 31 is connected to the distal-end constituting portion 21 of the distal end portion 6. The proximal-most joint ring 36 is connected to a tubular body such as a spiral tube, not shown, provided in the flexible tube portion 8.

The bending tube 30 is covered with a bending rubber 40 which is an outer cover. The bending rubber 40 includes, on the inner circumferential side thereof, a braid, not shown, such as a metal braided tube. The distal end part of the bending rubber 40 is secured to the outer circumference of the distal-end constituting portion 21, by a thread-wound adhesion portion, not shown, and the like.

The insertion portion 2 includes inside thereof a plurality of long constituent elements which are internal components. Specifically, in the present embodiment, the insertion portion 2 includes, in the inside thereof, four bending operation wires 44 which are long members for operating to bend the bending portion 7.

In addition, inside the insertion portion 2, long members, such as the light guide bundle, an image pickup cable, and various kinds of channels (none of them are shown) are inserted. Note that these long members are inserted from the insertion portion 2 through to the operation portion.

The respective distal end parts of the four bending operation wires 44 are fixed to wire fastening members 41 in the distal-end constituting portion 21. The four bending operation wires 44 are operated to be pulled or relaxed, to thereby cause the bending tube 30 to move rotationally around the respective joint portions 37. With such an operation, the bending portion 7 is bent in the up, down, left, or right direction on an observation screen.

In other words, the bending tube 30 includes a plurality of joint portions 37 for bending the bending tube 30 in a direction including the up/down direction as a first directional component orthogonal to the longitudinal axis X and a plurality of joint portions 37 for bending the bending tube 30 in a direction including the left/right direction as a second directional component orthogonal to the longitudinal axis X are alternately provided.

Note that the respective joint rings 32, 33, 35, and 36, except for the distal-most joint ring 31 and the respective first middle joint rings 34, include, on the inner circumferential surfaces thereof, a plurality of wire guides 42 and 43 that are joined to the inner circumferential surfaces and serve as wire receivers through which the bending operation wires 44 are respectively inserted. The plurality of wire guides 42 and 43 are guiding members configured to guide the bending operation wires 44 which are long members.

Here, a manufacturing method of the bending tube 30 disposed in the bending portion 7 will be described below. Note that the bending tube 30 in the present embodiment is mass-produced by automatic press working by using a press machine and a press feeding apparatus that are dedicated therefor.

Specifically, the bending tube 30 is manufactured by a sheet fabricating process with a progressive press working, and a layering process, a contour cutting process, and a bending process, with a transfer working.

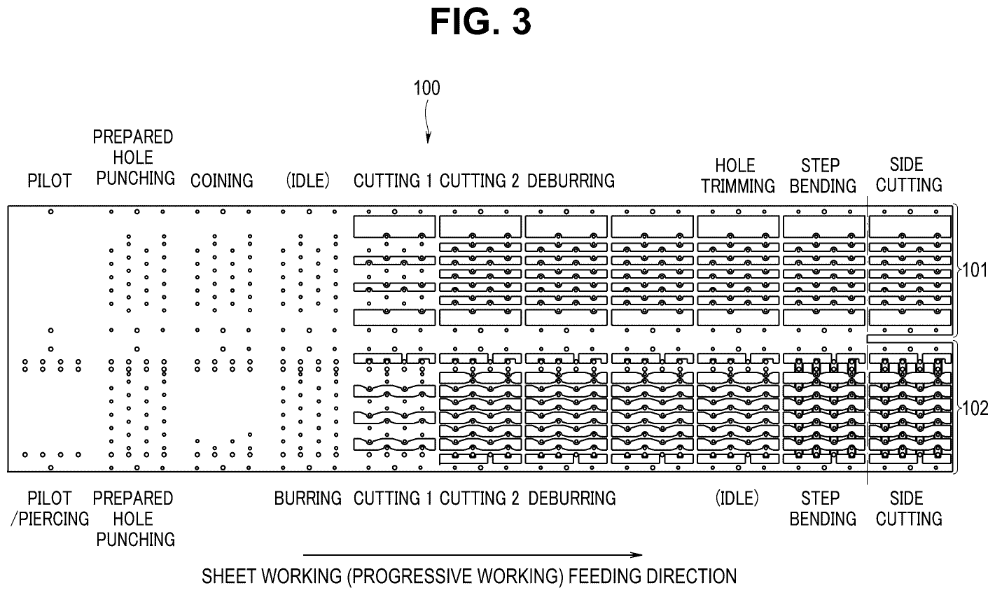

As shown in FIG. 3, the sheet fabricating processing is performed by pressing a coil member of a sheet material made of metal such as stainless steel having 0.2 mm to 1.0 mm thickness, for example, by a press machine, not shown. In this sheet fabricating processing, a working sheet 100 made of the sheet material that has gone through a plurality of processing stages is fabricated. Note that the plurality of processing stages include positioning with pilots, piercing, prepared hole punching, coining, burring, cutting (1, 2), deburring, hole trimming, step bending, side cutting, and the like.

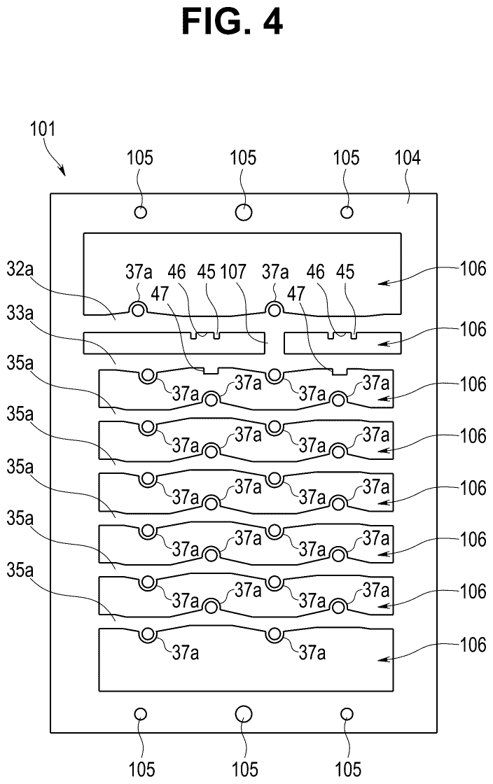

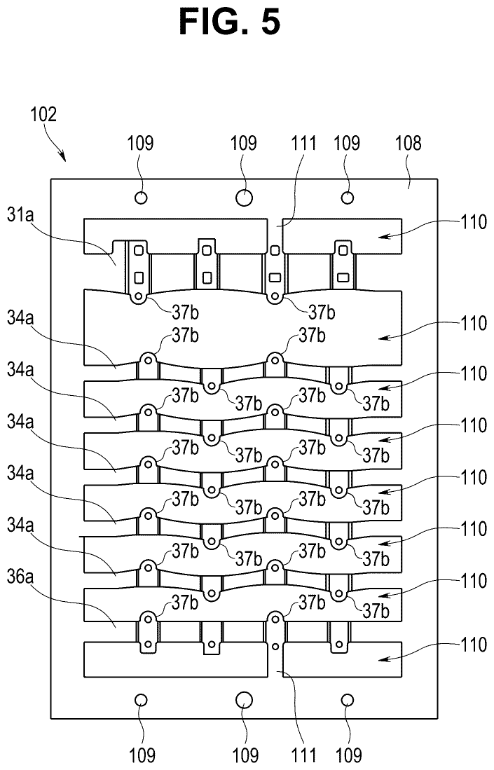

The working sheet 100 thus manufactured is separated into two sheet members, i.e., a first press working sheet 101 shown in FIG. 4 and a second press working sheet 102 shown in FIG. 5 in the final cutting process in the progressive press working. In other words, the first press working sheet 101 and the second press working sheet 102 are simultaneously fabricated from the same metal coil member.

As shown in FIG. 4, in the first press working sheet 101, a plurality of joint ring preparatory bodies 32a, 33a, 35a, and the like are formed. The plurality of joint ring preparatory bodies 32a, 33a, and 35a each include arc-shaped first protrusion portions 37a, each of which serves as a pivotal mechanism of each of the joint portions 37.

In the first press working sheet 101, a plurality of pilot holes 105 and a plurality of blanking portions 106 are formed. The respective pilot holes 105 are formed, by punching and the like, in a carrier 104, which serves as a feed pier of the first press working sheet 101. The respective pilot holes 105 are holes into which pilot pins, not shown, which define pressing positions of the first press working sheet 101, are inserted.

The respective blanking portions 106 are parts formed by the metal coil member being hollowed out with a die, not shown, in accordance with the contours of the first linking joint ring preparatory body 32a, the second linking joint ring preparatory body 33a, the (second) middle joint ring preparatory body 35a, and the like, as the plurality of the joint ring preparatory bodies.

The first linking joint ring preparatory body 32a is a base material that later becomes the first linking joint ring 32. The first linking joint ring preparatory body 32a includes a pair of first joint-welding portions 45 formed in the up/down direction in which the bending portion 7 bends. The respective first joint-welding portions 45 are formed so as to protrude toward the proximal end side from one edge of the first linking joint ring preparatory body 32a (the one edge being opposite to the second linking joint ring preparatory body 33a to be described later).

Each of the first joint-welding portions 45 includes, at the center thereof, a recessed portion 46. In other words, each of the first joint-welding portions 45 has a U-shape protruded toward the proximal end side.

The second linking joint ring preparatory body 33a is a base material that later becomes the second linking joint ring 33. The second linking joint ring preparatory body 33a includes a pair of second joint-welding portions 47 formed in the up/down direction in which the bending portion 7 bends. Each of the second joint-welding portions 47 is formed so as to protrude toward the proximal end side from an edge of the second linking joint ring preparatory body 33a, the edge being opposite to the second middle joint ring preparatory body 35a.

Each of the second joint-welding portions 47 is set to be a shape having a dimension fitted to each of the recessed portions 46 of the first joint-welding portions 45. Each of the second joint-welding portions 47 is a bent portion that is bent at substantially a right angle (≈) 90° in a radially outward direction of the second linking joint ring 33 by a bending processing to be described later. Note that the (second) middle joint ring preparatory body 35a is a base material that later becomes the second middle joint ring 35.

As shown in FIG. 5, in the second press working sheet 102, a plurality of joint ring preparatory bodies 31a, 34a, 36a, and the like are formed.

The plurality of joint ring preparatory bodies 31a, 34a, and 36a each include arc-shaped second protrusion portions 37b which serve as the pivotal mechanism of each of the joint portions 37. Note that the first linking joint ring preparatory body 32a and the second linking joint ring preparatory body 33a are in the state connected to each other by a bridge 107. The bridge 107 is an edge pier as a connecting portion to be separated later.

Also in the second press working sheet 102, a plurality of pilot holes 109 and a plurality of blanking portions 110 are formed. The respective pilot holes 109 are also formed, by punching and the like, in a carrier 108, which serves as a feed pier of the second press working sheet 102. The respective pilot holes 109 are also holes into which pilot pins, not shown, which define pressing positions of the second press working sheet 102, are inserted.

The respective blanking portions 110 are also parts formed by the metal coil member being hollowed out by a die, not shown, in accordance with the contours of the distal-most joint ring preparatory body 31a, the first middle joint ring preparatory body 34a, and the proximal-most joint ring preparatory body 36a, as the plurality of the joint ring preparatory bodies.

The distal-most joint ring preparatory body 31a is a base material that later becomes the distal-most joint ring 31. The distal-most joint ring preparatory body 31a is in the state connected to the carrier 108 by a bridge 111. The bridge 111 is also an edge pier as a connecting portion to be separated later.

The first middle joint ring preparatory body 34a is a base material that later becomes the first middle joint ring 34. The proximal-most joint ring preparatory body 36a is a base material that later becomes the proximal-most joint ring 36. The proximal-most joint ring preparatory body 36a is in the state connected to the carrier 108 by the bridge 111 as a connecting portion to be separated later.

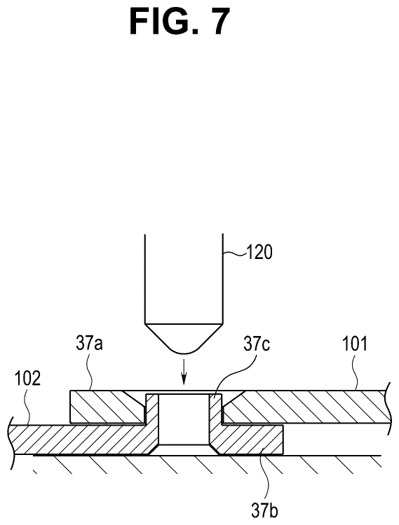

Note that the respective second protrusion portions 37b, which are formed in the second press working sheet 102, each include a burring portion 37c (see FIG. 7). The burring portion 37c is an annular portion protruded from one surface of each of the second protrusion portions 37b by performing press working on the prepared hole of each of the second protrusion portions 37b in a predetermined direction (upper direction).

Next, the two second joint-welding portions 47 of the second linking joint ring 33 are subjected to press working, to be bent at substantially the right angle (90°) in a surface direction which is the radially outward side of the bending tube 30. In other words, each of the second joint-welding portions 47 is formed by the protrusion portion, which is extended from the end portion of the second linking joint ring 33, being bent in a direction away from the longitudinal axis X.

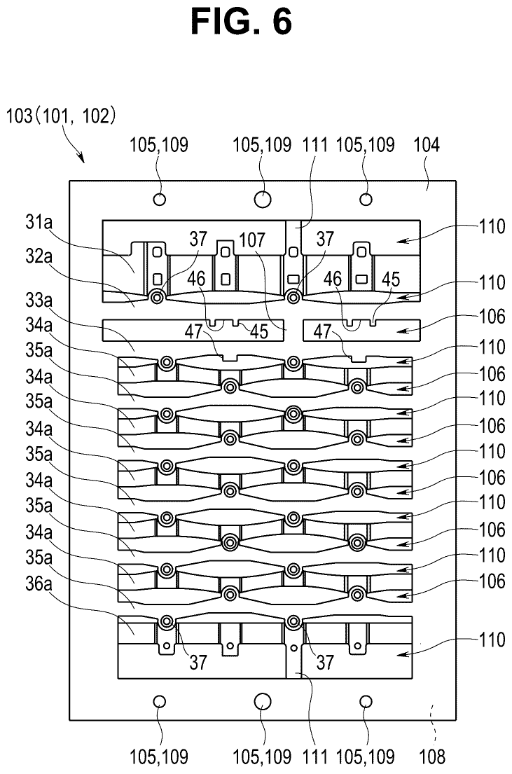

The first press working sheet 101 and the second press working sheet 102 that have been thus fabricated are placed such that the first press working sheet 101 is layered on the second press working sheet 102, as shown in FIG. 6. Thus, a bending tube processing sheet 103 is fabricated. At this time, the burring portion 37c of each of the second protrusion portions 37b in the second press working sheet 102 is inserted into a hole portion of each of the protrusion portions 37a in the first press working sheet 101.

Note that the layering process of the first press working sheet 101 and the second press working sheet 102 is performed by the sheets being automatically transferred by the press feeding apparatus. Furthermore, the bending tube processing sheet 103 is transferred to various pressing processes such as the punching process, the bending process, etc., by a transfer apparatus, and press-worked.

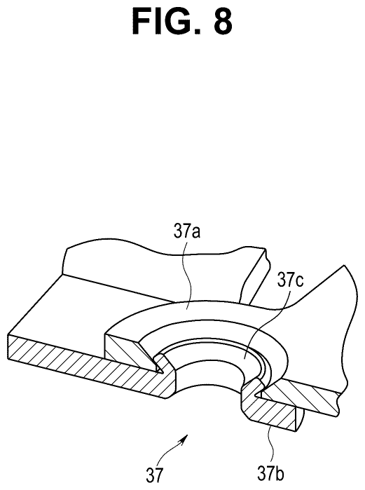

As shown in FIG. 7, the bending tube processing sheet 103 is subjected to a pressing process by a press jig 120, to cause the burring portion 37c of each of the second protrusion portions 37b to expand on the first protrusion portion 37a into which the burring portion 37c is inserted. Thus, the respective joint portions 37 as shown in FIG. 8 are formed in the bending tube processing sheet 103. Thereby, the respective joint ring preparatory bodies 31a, 32a, 33a, 34a, 35a, and 36a are coupled with each other.

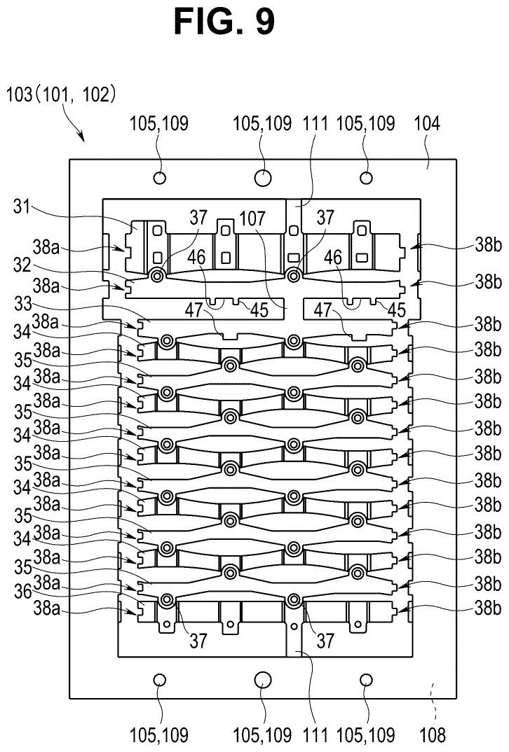

Then, as shown in FIG. 9, the bending tube processing sheet 103 is press-worked such that the both side portions of the respective joint ring preparatory bodies 31a, 32a, 33a, 34a, 35a, and 36a are cut to be separated from the carriers 104 and 108. At this time, the both side portions of each of the joint ring preparatory bodies 31a, 32a, 33a, 34a, 35a, and 36a are subjected to cutout (notch punching) processing such that a first joining end portion 38a in a recessed shape and a second joining end portion 38b in a protruded shape are formed.

Thus, in the bending tube processing sheet 103, the base materials of developed shapes of the respective joint rings 31, 32, 33, 34, 35, and 36 are formed. Note that the bridge 107 that connects the first linking joint ring 32 and the second linking joint ring 33 and the bridge 111 that connects the distal-most joint ring 31 or the proximal-most joint ring 36 and the carrier 108 are left.

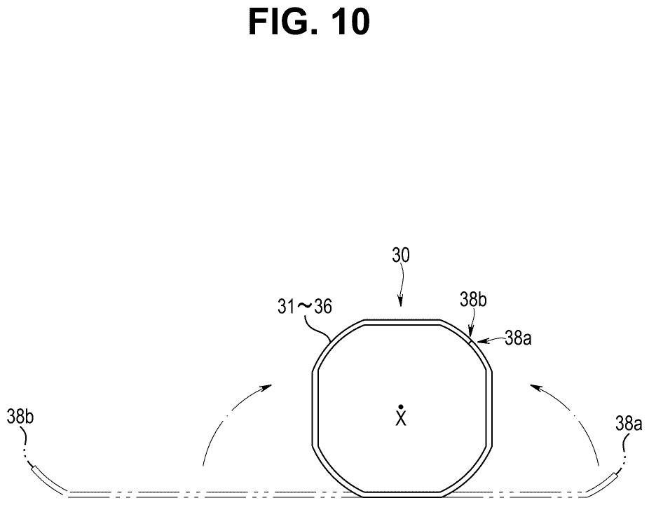

Then, as shown in FIG. 10, each of the respective joint rings 31, 32, 33, 34, 35, and 36 is subjected to bending processing (rounding processing) to be a cylindrical surface with an approximate radius of curvature R6, such that each of the first joining end portions 38a and each of the second joining end portions 38b are fitted to each other.

Note that each of the joint rings 31, 32, 33, 34, 35, and 36 is configured such that the diameter dimension φ of the outer diameter (diameter) of each of the joint rings is set to a range from a small diameter of 6.0 mm to a large diameter of 13.0 mm, for example, depending on a plate thickness (0.2 mm to 0.5 mm).

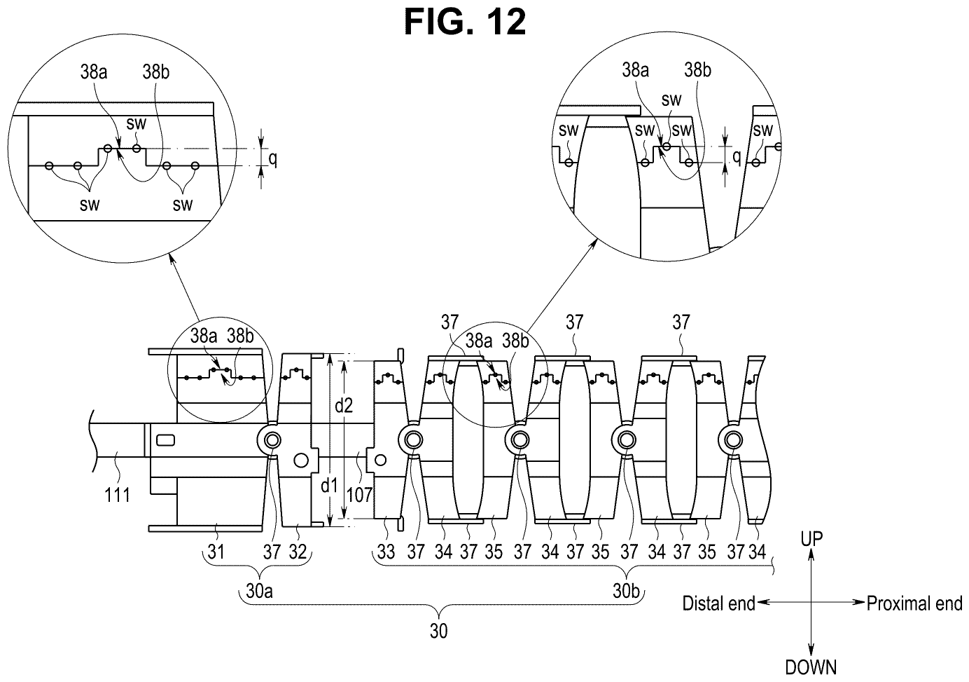

In each of the joint rings 31, 32, 33, 34, 35, and 36 subjected to the bending processing, as shown in FIG. 12, each of the first joining end portions 38a and each of the second joining end portions 38b, which are fitted to each other, are spot-welded SW by laser-welding and the like. In other words, each of the first joining end portions 38a and each of the second joining end portions 38b are fitted to each other at the parts facing each other, to be welded by the spot-welding SW.

The first joint ring 32 and the second joint ring 33 are formed from the sheet material.

At this time, the respective end surfaces of either one of the respective first joining end portions 38a or the respective second joining end portions 38b become abutment portions against which the respective end surfaces of the other of the respective first joining end portions 38a or the respective second joining end portions 38b abut. The respective end surfaces serving as the abutment portions are along the longitudinal axis X direction of the first joint ring group 30a and the second joint ring group 30b. Then, the respective abutment portions are joined by the spot-welding SW. Note that the respective end surfaces at which each of the first joining end portions 38a and each of the second joining end portions 38b abut against each other are planes in a thickness direction of each of the first joining end portions 38a and each of the second joining end portions 38b.

Note that each of the first joining end portions 38a and each of the second joining end portions 38b has a protruded/recessed shape with a seam q of 1 mm or less, in order to improve assemblability. In addition, the spot-welding SW is performed on the joining portion of one first joining end portion 38a and one second joining end portion 38b at three to seven points, for example.

Thus, in the bending tube processing sheet 103, the first joint ring group 30a in which the distal-most joint ring 31 and the first linking joint ring 32 are coupled by the joint portion 37 and the second joint ring group 30b in which the second linking joint ring 33, the plurality of middle joint rings 34, 35, and the proximal-most joint ring 36 are coupled by the joint portions 37 are formed.

Note that an outer diameter d1 of the first linking joint ring 32 of the first joint ring group 30a is set to be larger than an outer diameter d2 of the second linking joint ring 33 of the second joint ring group 30b (d1>d2). In other words, in the bending tube processing sheet 103 in the present embodiment, the first joint ring group 30a constituting an anterior joint having a large diameter and the second joint ring group 30b constituting a posterior joint having a small diameter in the bending tube 30 are formed by press working.

Then, the first joint ring group 30a and the second joint ring group 30b are separated from the bending tube processing sheet 103, to be separated from each other. After that, wire guides 42 and 43, not shown, are laser-welded to the first joint ring group 30a and the second joint ring group 30b from the direction of the outer circumferential portions of the first and second joint ring groups 30a, 30b. Note that the laser-welding of the wire guides 42 and 43 may be performed simultaneously with the spot-welding SW by laser of the joining portion of each of the first joining end portions 38a and each of the second joining end portions 38b. In addition, the wire guides 42 and 43 may be fixed to the first joint ring group 30a and the second joint ring group 30b by brazing, adhesion, or the like.

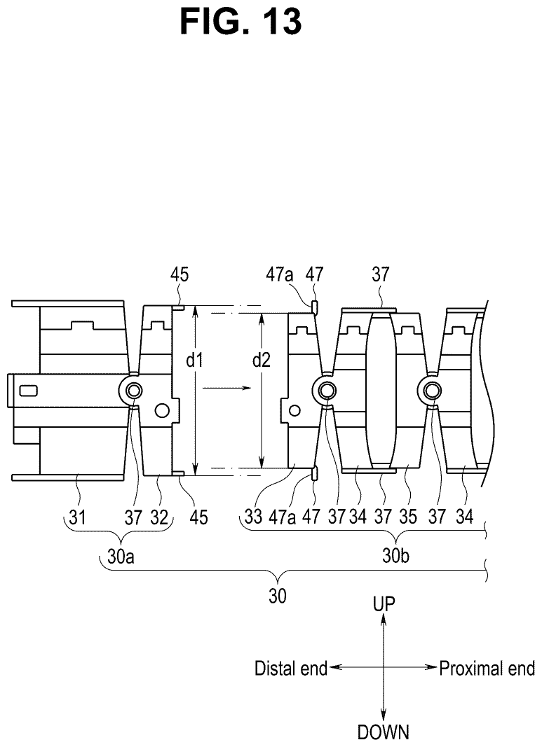

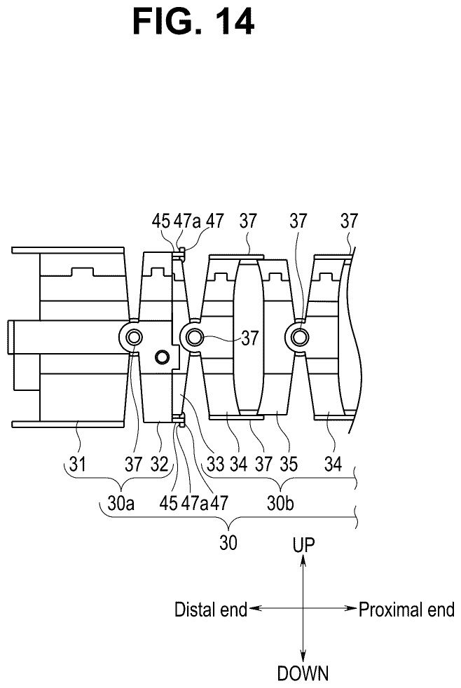

The first joint ring group 30a and the second joint ring group 30b are assembled to each other after the wire guides 42 and 43 are welded. Specifically, as shown in FIG. 13 and FIG. 14, the first linking joint ring 32 of the first joint ring group 30a and the second linking joint ring 33 of the second joint ring group 30b are joined. The first linking joint ring 32 is joined such that the proximal end part thereof covers the distal end part of the second linking joint ring 33.

A proximal end portion of the first joint ring 32 can be overlapped on a distal end portion of the second joint ring 33. The first joint ring 32 can have the first diameter d1 centered about a longitudinal axis X, the second joint ring 33 can have the second diameter d2 centered about the longitudinal axis. The first and the second joint rings 32, 33 can be formed such that the first diameter d1 is larger than the second diameter d2.

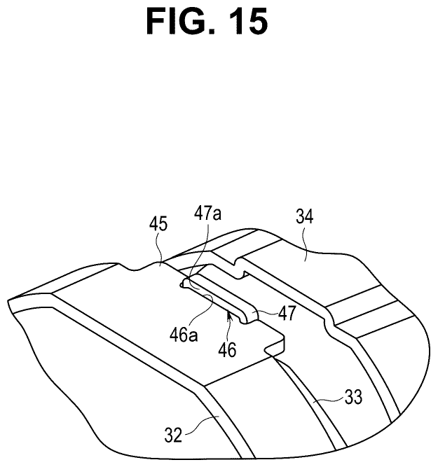

At this time, an assembly jig, not shown, is used, to cause the upper and lower pair of second joint-welding portions 47 in the bending direction of the bending portion 7 to be fitted into the recessed portions 46 of the upper and lower pair of first joint-welding portions 45, as shown in FIG. 15. Note that each of the second joint-welding portions 47 becomes an abutting portion formed such that an end surface 47a on the distal end side, which is bent at substantially the right angle (˜) 90° in the radially outward direction of the first linking joint ring 32, abuts against a proximal end surface 46a which is an edge portion of the recessed portion 46 of each of the first joint-welding portions 45. In other words, each of the second joint-welding portions 47 abuts against the surface of the end portion in the direction of the longitudinal axis of the first linking joint ring 32.

The abutting portion in the second joint ring 33 can be configured to abut against an end surface of the first joint ring 32, and the abutting portion can extend in a direction intersecting the longitudinal axis X. The abutting portion can be joined to the end surface of the first joint ring 32. The first and the second joint rings 32, 33 can be formed a punching process and a rounding process. The abutting portion can be joined to the end surface includes by laser-welding. The laser-welding can be performed by applying a laser, at a predetermined angle θ inclined with respect to the longitudinal axis X, to a contact part of the abutting portion and the end surface.

The first and the second joint rings 32, 33 each can include a recess and a protrusion, and the recesses and the protrusions can be joined by spot-welding.

Each of the second joint-welding portions 47 as the abutting portions is formed at a position shifted with respect to the position of the joint portion 37 closest to the first linking joint ring 32 against which each of the second joint-welding portions 47 abuts, by a predetermined angle, i.e., substantially 90° in the present embodiment, about the longitudinal axis X as the center axis. In addition, each of the second joint-welding portions 47 is provided in the up/down direction which is the side where the bending portion 7 is bent at the largest angle with the longitudinal axis X as the center.

Then, each of the first joint-welding portions 45 and each of the second joint-welding portions 47 are joined by the laser-welding LW. At this time, each of the first joint-welding portions 45 and each of the second joint-welding portions 47 configure the abutting portions at which the proximal end surface 46a and the end surface 47a abut against each other. The proximal end surface 46a and the end surface 47a are opposed surfaces of the end portions in the direction of the longitudinal axis of the first joint ring group 30a or the second joint ring group 30b. Then, the respective abutting portions are joined by the laser-welding LW. Note that the proximal end surface 46a and the end surface 47a are orthogonal planes to the longitudinal axis X of the bending tube 30 in which the first joint ring group 30a and the second joint ring group 30b are joined.

Thus, the bending tube 30 in the present embodiment has a structure in which the first linking joint ring 32 of the first joint ring group 30a and the second linking joint ring 33 of the second joint ring group 30b, which differ in diameter, are joined by the laser-welding LW.

Note that each of the first joint-welding portions 45 and each of the second joint-welding portions 47 may be joined by the spot-welding SW, adhesion, and the like.

Thus, the first linking joint ring 32 and the second linking joint ring 33 are integrated into one joint ring. Thereby, the bending tube 30 is configured in which the first joint ring group 30a having the large diameter, which is on the distal end side of the bending tube, and the second joint ring group 30b having the small diameter, which is on the proximal end side of the bending tube are joined.

Note that, at the time of the laser-welding LW of each of the first joint-welding portions 45 and each of the second joint-welding portions 47, the second joint-welding portions 47 are fitted respectively into the recessed portions 46 of the two, i.e., upper and lower first joint-welding portions 45 in the bending direction of the bending portion 7.

With such a configuration, the first linking joint ring 32 and the second linking joint ring 33 are configured such that the positions in the left/right direction are restricted when the bending portion 7 is bent. Furthermore, the first linking joint ring 32 and the second linking joint ring 33 are configured such that the longitudinal axes X, which are the center axes thereof, are made to be coincided with each other, by the assembly jig, not shown, and the positions in the up/down direction are restricted when the bending portion 7 is bent.

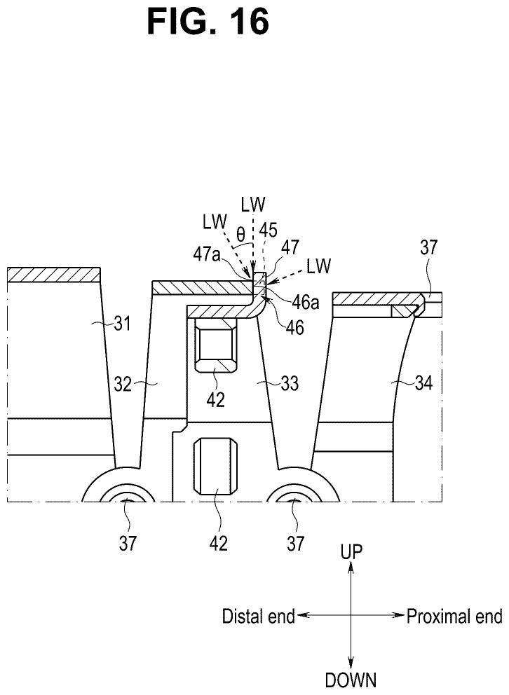

As shown in FIG. 16, the laser-welding LW is performed from the outer diameter side of the second joint-welding portion 47 toward the ridge line of the recessed portion 46 of the first joint-welding portion 45 against which the second joint-welding portion 47 abuts in a contact manner, that is, the laser-welding LW is performed on the surface of the first joint-welding portion 45 in substantially the vertical direction. Note that the laser-welding LW may be performed in an oblique direction having a predetermined angle θ at which the laser is allowed to penetrate through the second joint-welding portion 47 or may be performed from the rear surface side of the second joint-welding portion 47. The laser-welding LW for joining the first joint-welding portion 45 and the second joint-welding portion 47 may be automatically performed by a dedicated laser welding apparatus.

The protrusion 47 can be formed at the abutting portion by bending an end portion of the second joint ring 33 in a direction away from the longitudinal axis X. A radially distal end of the abutting portion can be radially outward of an outer circumference surface of the first joint ring 32.

Thus, as a method of joining the first linking joint ring 32 and the second linking joint ring 33 that have different diameters, the joining by the laser-welding LW is used, to thereby be capable of suppressing a decrease in the volume of the internal space (pressure to the internal components) of the bending tube 30, compared with the joining by swaging which is often used in the press working.

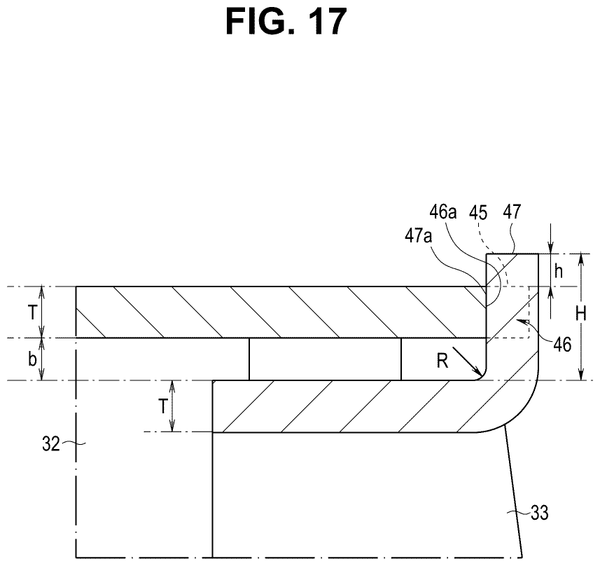

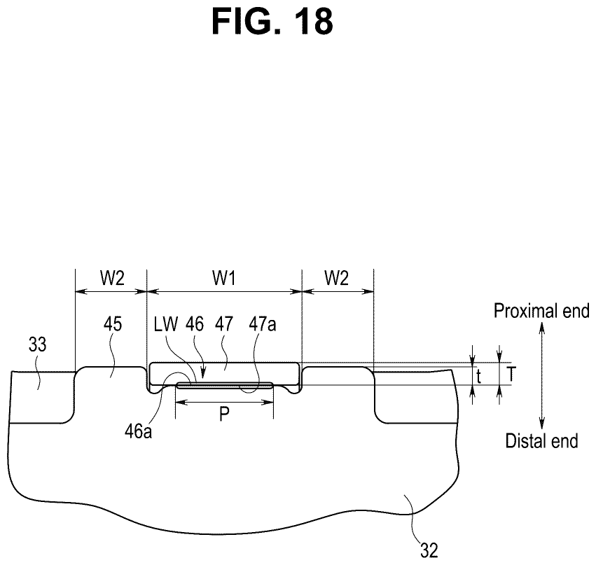

Here, description will be made on the dimensional relationship, and the like, between the first joint-welding portion 45 of the first linking joint ring 32 and the second joint-welding portion 47 of the second linking joint ring 33. First, as shown in FIG. 17 and FIG. 18, the plate thickness T of each of the first linking joint ring 32 and the second linking joint ring 33 is 0.2 mm to 0.5 mm (T=0.2 to 0.5), for example. In addition, the diameter dimension q of the bending tube 30 is set to a range from the small diameter of 6 mm (φ=6.0) to the large diameter of 13 mm (φ=13.0).

As shown in FIG. 17, a gap b between the first linking joint ring 32 and the second linking joint ring 33 is set to be 0.25 mm (b=0.25 mm), for example. In addition, the inner radius of curvature R of the second joint-welding portion 47, which is bent at substantially the right angle (≈) 90° in the radially outward direction of the second linking joint ring 33, is set to be 0.1 mm (R=0.1 mm), for example.

The inner circumference of the first joint ring 32 can be spaced apart from the outer circumference of the second joint ring 33 in a radial direction of the first joint ring 32.

In this case, a height H of a rising of the second joint-welding portion 47 is set to a dimension equal to or larger than 2T+R, i.e., a sum of twice the plate thickness T of each of the first linking joint ring 32 and the second linking joint ring 33 and the radius of curvature R (H≥2T+R). If the plate thickness T of each of the first linking joint ring 32 and the second linking joint ring 33 is 0.3 mm (T=0.3), the height H of the rising of the second joint-welding portion 47 is set to be 0.7 mm or more, for example, 0.75 mm (H=0.75 mm).

In such a dimensional relationship, the second joint-welding portion 47 has the height H protruding from the surface of the first joint-welding portion 45. When the height H of the second joint-welding portion 47 is set to be 0.75 mm (H=0.75 mm), the protruding amount h is 0.2 mm (h=0.2). Note that the protruding amount h is set to a range from 0 mm to 0.5 mm (0≤h≤0.5).

Even if the joining position of the first linking joint ring 32 and the second linking joint ring 33 shifts in the up/down direction, the protruding amount h of the second joint-welding portion 47 satisfies the dimensional condition in which the protruding amount h is larger than the difference obtained by subtracting the inner radius of curvature R of the second joint-welding portion 47 from the gap b between the first linking joint ring 32 and the second linking joint ring 33 (h>b−R). With such a dimensional condition, the welding area of the second joint-welding portion 47 to the first joint-welding portion 45 is secured.

Thus, the height H of the second joint-welding portion 47 is set to the dimension slightly protruding from the first joint-welding portion 45 in the radially outward direction so that the laser-welding LW can be performed easily. If the second joint-welding portion 47 has no protruding allowance, the thickness of the second joint-welding portion 47 is not enough for the laser-welding LW. In view of this, at the time of the press forming, the second joint-welding portion 47 may be protruded with respect to the surface position of the first joint-welding portion 45.

In addition, since the edge of the second joint-welding portion 47 is rounded when the laser-welding LW is applied, there is no risk of damaging the bending rubber 40 covering the bending tube 30. In other words, the protruding height of the second joint-welding portion 47 is reduced to make the cross-section thereof round by the laser-welding LW, which reduces the risk of damaging the bending rubber 40.

As shown in FIG. 18, a width W1 of the recessed portion 46 of the first joint-welding portion 45 is set to be 2.0 mm (W1=2.0), for example. In addition, widths W2 of the both side portions, across the recessed portion 46, of the first joint-welding portion 45 are set to be the same dimension, for example, 1.0 mm (W2=1.0). In other words, the width W1 of the recessed portion 46 formed in the first joint-welding portion 45 is twice the width W2 of the both side portions (W1=2W2).

If the plate thickness T of the second joint-welding portion 47 is 0.3 mm (T=0.3), a depth t of the recessed portion 46 formed in the first joint-welding portion 45 is 0.25 mm, for example. In other words, the depth t of the recessed portion 46 has a dimension shorter, by 0.05 mm, than the plate thickness T (0.3 mm) of the second joint-welding portion 47 to be fitted into the recessed portion 46 (T>t). With such a dimension relationship, the second joint-welding portion 47 protrudes toward the proximal end side with respect to the first joint-welding portion 45.

If the width W1 of the recessed portion 46 of the first joint-welding portion 45 is 2.0 mm (W1=2.0), for example, a range P of the laser-welding LW for joining the first joint-welding portion 45 and the second joint-welding portion 47 is set to be about 1.4 mm (P≈1.4), for example. The laser-welding LW is set to a pulse width of 0.5 to 10 msec with an output of 400 to 800 watts (W), as the conditions.

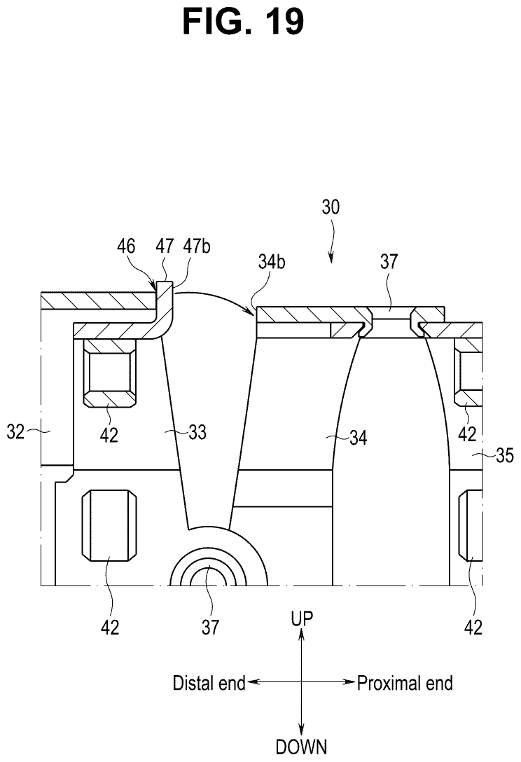

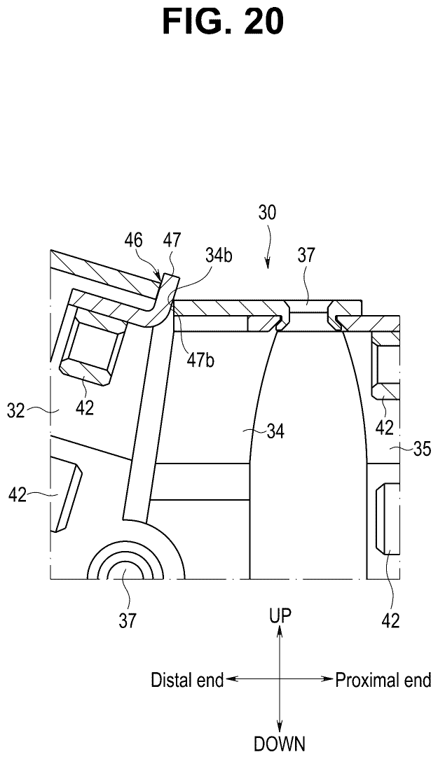

The second joint-welding portion 47 protrudes toward the proximal end side with respect to the first joint-welding portion 45, to thereby cause the end surface 47b on the proximal end side to surely abut against the end surface 34b on the distal end side of the first middle joint ring 34 when the bending portion 7 is bent maximally, as shown in FIG. 19 and FIG. 20. Such a configuration improves the accuracy of the bending function of the bending portion 7.

The first and the second joint rings 32, 33 can form a joint ring pair 32, 33 that can be configured to pivot relative to a joint portion 37 of a third joint ring 34 about a pivot axis at a bending angle, and a circumferential position of the abutting portion is offset in a circumferential direction relative to a circumferential position of the joint portion 37.

The circumferential position of the abutting portion can coincide with a location at which an arc length of the bending angle separating the third joint ring 34 and the joint ring pair 32, 33 is a maximum.

The bending tube for use with an insertion instrument can comprises the joint ring pair including: the first joint ring 32 including an end surface, the second joint ring 33 including the abutting portion configured to be fixed to the end surface of the first joint ring in the longitudinal direction of the joint ring pair. The abutting portion can extend in a direction intersecting the longitudinal direction.

The third joint ring 34 can be configured to pivot relative to the joint ring pair via the joint portion 37. The abutting portion can include: a first surface configured to fixed to the end surface of the first joint ring 32; and a second surface configured to contact an end surface of the third joint ring 34 when the joint ring pair pivots relative to the third joint ring.

Note that, in the above-described manufacturing method of the bending tube 30, before joining the first joint ring group 30a having the large diameter and the second joint ring group 30b having the small diameter, the respective wire guides 42 and 43 may be joined to the first joint ring group 30a and the second joint ring group 30b first by the laser-welding LW.

After the respective wire guides 42 and 43 are disposed in the first joint ring group 30a and the second joint ring group 30b, the first joint-welding portion 45 and the second joint-welding portion 47 are joined by the laser-welding LW. This is because, if the first joint ring group 30a and the second joint ring group 30b, which differ in diameter, are joined first by the laser-welding LW, sites would be created where it is difficult to join the wire guides 42 by the laser-welding LW.

The guide member 42, 43 can be joined to the inner surface of the first and the second joint rings 32, 33 by laser-welding. The guide member 42, 43 can be configured to guide a long member (wire) inserted through each of the first and the second joint rings 32, 33.

In the above-described method, the plate member is press-worked to fabricate the press working sheets 101, 102, these sheets are layered to form the bending tube processing sheet 103, and while performing the bending processing (rounding processing) on the bending tube processing sheet 103, the joint rings 31, 32, 33, 34, 35, and 36 are each processed such that the joining end portions are fitted to each other to form the cylindrical surface. In such a method, the first joint ring group 30a and the second joint ring group 30b, which differ in diameter, can be created simultaneously, if the first joint ring group 30a and the second joint ring group 30b are in the state not connected to each other.

However, in the joint ring group (the joint ring group in which a part corresponding to the first joint ring group 30a and a part corresponding to the second joint ring group 30b are connected in series), the diameter changes significantly in the middle thereof. Therefore, it is very difficult to perform the bending processing (rounding processing) on the joint ring group such that all the joining end portions of the respective joint rings 31, 32, 33, 34, 35, and 36 are fitted to each other, with the longitudinal axis X being in common in the part corresponding to the first joint ring group 30a and the part corresponding to the second joint ring group 30b.

In addition, the joint ring group (joint ring group in which the part corresponding to the first joint ring group 30a and the part corresponding to the second joint ring group 30b are connected in series), the diameter of which changes significantly in the middle, can be press-worked by dividing the processing steps into several smaller steps and adding a plurality of additional steps. However, in such a case, the number of the processing steps increases greatly, which results in a large increase in the manufacturing cost.

In view of the above, in the manufacturing method of the bending tube 30 in the present embodiment, the first joint ring group 30a and the second joint ring group 30b, which differ in diameter, are press-worked (transfer-worked) to be separated from the bending tube processing sheet 103, and thereafter the first joint ring group 30a and the second joint ring group 30b are joined.

In addition, the spot-welding SW for joining the first joining end portion 38a and the protruded-shaped second joining end portion 38b of each of the joint rings 31, 32, 33, 34, 35, and 36 can be performed by the laser-welding LW. Thus, the laser welding apparatus can be commonly used in the spot-welding SW, and in the laser-welding LW for joining the first joint ring group 30a and the second joint ring group 30b, which enables the welding processes to be integrated.

In the above-described manufacturing method of the bending tube 30, by taking the mass productivity including the cost into consideration, the two sheets, i.e., the first press working sheet 101 and the second press working sheet 102 in which the developed base materials of the plurality of joint rings 31, 32, 33, 34, 35, and 36 are formed by pressing are fabricated from the coil member which is the same metal sheet material.

In the manufacturing method of the bending tube 30, the first press working sheet 101 and the second press working sheet 102 are layered, and the joint portions 37 that rotatably couple each of the plurality of joint rings 31, 32, 33, 34, 35, and 36 are press-worked.

Next, in the manufacturing method of the bending tube 30, the plurality of joint rings 31, 32, 33, 34, 35, and 36 are subjected to the rounding processing by pressing, to form the first joint ring group 30a and the second joint ring group 30b, which differ in diameter, in the first press working sheet 101 and the second press working sheet 102 that are sheet materials.

After that, in the manufacturing method of the bending tube 30, in another process, the first joint ring group 30a and the second joint ring group 30b, which differ in diameter, are joined. Note that the bending tube 30 in the present embodiment is a structural body having the diameter change in the first joint ring group 30a having the large diameter and the second joint ring group 30b having the small diameter.

In addition, the bending tube 30 includes the joint portions 37 provided continuously at short intervals in the same direction, and the first joint-welding portions 45 and the second joint-welding portions 47 are provided in the up/down direction so as to enable the distal end side of the bending tube 30 to be bent largely in the up/down direction. The first joint-welding portions 45 and the second joint-welding portions 47 are the connecting parts at which the first linking joint ring 32 and the second linking joint ring 33, which are joint rings having different diameters, are joined by welding. Furthermore, in the bending tube 30, the number of the joint portions 37 provided on the left and right sides is increased so that the bending angle of the bending tube 30 in the up/down direction becomes large.

As described above, the bending tube 30 is manufactured by the method using the press working, which can eliminate the restrictions on forming due to a diameter expansion processing such as a bulge forming, and achieve a desired expanded diameter structure with one joint ring formed by joining the first linking joint ring 32 and the second linking joint ring 33.

Conventionally, in order to fabricate the bending tube 30 having the diameter change by coupling the plurality of joint rings, diameter expansion processing had to be performed on the plurality of joint rings with a difficult forming such as the bulge forming.

In the bending tube which has the diameter change, due to the restrictions on forming, the diameter of each of the plurality of joint rings has to be changed. Therefore, in manufacturing the bending tube which has the diameter change, the kinds of processing increase, which requires a large number of processes. In other words, in order to manufacture the bending tube which has the diameter change, the manufacturing cost rises according to the man-hour for processing.

Furthermore, it is difficult to fabricate the bending tube 30 which has the diameter change by the conventional automatic press working. In contrast, the bending tube 30 in the present embodiment can be manufactured at a low cost by simplifying the manufacturing method by using the press working and the laser joint welding and suppressing the rise in the manufacturing cost. Thus, the bending tube 30 in the present embodiment is used for the single-use type endoscope 1 for which a low price is desired, to thereby contribute to the reduction in the manufacturing cost.

A single-use endoscope can be disposed of (discarded) after being used once, and should not be used multiple times.

The single-use endoscope can include a single-use component and a multi-use component. The single-use component is disposed or returned to manufacturer of after being used once, and the multi-use component is a reusable component that is repeatedly used.

Even the reusable endoscope capable of being used multiple times by being reprocessed can be stopped being used and be sent to the manufacturer or the like for maintenance after being used a predetermined number of times.

In addition, the bending tube 30 in the present embodiment has a structure including the diameter change in each of the joint rings 31, 32, 33, 34, 35, and 36, for example, in contrast to the conventional structure used in an existing endoscope 1. Such a structure is employed for reducing as much as possible the diameter of the bending portion 7 in which the bending tube 30 is incorporated, to improve the insertion performance of the insertion portion 2 of the endoscope 1.

However, the distal end portion 6 of the insertion portion 2 needs to have a certain thickness for housing the internal components. In the endoscope 1, if the diameter of the bending portion 7 is made equal to the diameter of the distal end portion 6 of the insertion portion 2, the diameter of the bending portion 7 unnecessarily increases. Such a configuration will cause a concern of the increase in the burden on the patient (subject to be examined), and the influences on the insertion performance, the observation performance, and the like of the insertion portion 2, in a medical procedure.

In addition, diameter reduction is desired for the insertion portion 2 of the endoscope 1 for improving the insertion performance into the subject to be examined. In view of the above, the bending tube 30 in the present embodiment has the structure in which the first joint ring group 30a, which is located on the distal end side and connected to the distal end portion 6, has the large diameter in accordance with the diameter of the distal end portion 6, and the second joint ring group 30b, which is connected to the proximal end of the first joint ring group 30a, has the small diameter.

Thus, the bending tube 30 in the present embodiment has the joint ring structure having the diameter change, to thereby be capable of improving the increase in the burden on the patient, and the influences on the insertion performance and the observation performance of the insertion portion 2 of the endoscope 1, as the insertion instrument.

As described above, according to the manufacturing method of the bending tube 30 and the structure of the bending tube 30 in the present embodiment, the manufacturing method of the bending tube 30 of the endoscope 1 as the insertion instrument having the diameter change is simplified to achieve the reduced cost, and the bending tube 30 can be fabricated at a low cost. With the method and the structure, the endoscope 1 or the like, as the insertion instrument, uses the bending tube 30 in the present embodiment in the bending portion 7 of the insertion portion 2, to thereby eliminate the increase in the burden on the subject to be examined such as the patient, and the influences on the insertion performance into the subject, the observation performance, and the like.

### First Modified Example

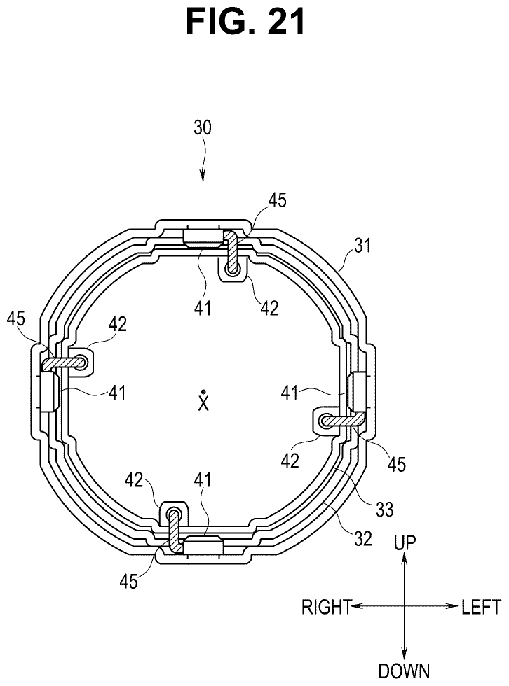

As shown in FIG. 21, four bending operation wires 44 may be configured such that the distal ends thereof are fixed, by swaging, respectively to four wire fastening members 41 joined to the distal-most joint ring 31 at up, down, left, and right positions about the longitudinal axis X of the bending tube 30. In other words, the four bending operation wires 44 are provided here, and each of the distal ends thereof are fixed individually to each of the wire fastening members 41. Note that the distal ends of the respective bending operation wires 44 may be fixed to the distal-most joint ring 31 by welding or brazing, instead of using the wire fastening members 41.

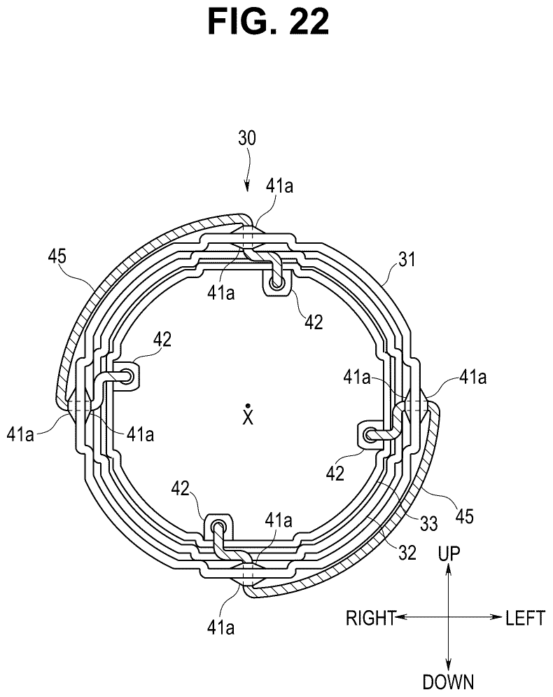

Note that, as shown in FIG. 22, two bending operation wires 44 may be provided and inserted through the distal-most joint ring 31, at an upper portion and a right portion, and at a lower portion and a left portion about the longitudinal axis X of the bending tube 30. Each of the respective bending operation wires 44 is disposed from the upper portion to the right portion or from the lower portion to the left portion, along the outer circumference of the distal-most joint ring 31, and the parts where the bending operation wires are inserted through the distal-most joint ring 31 are fixed by welding or brazing.

### Second Modified Example

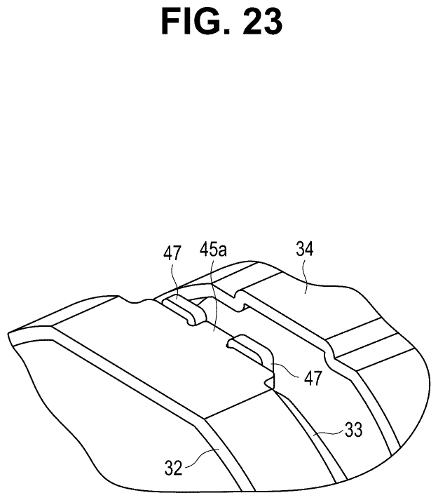

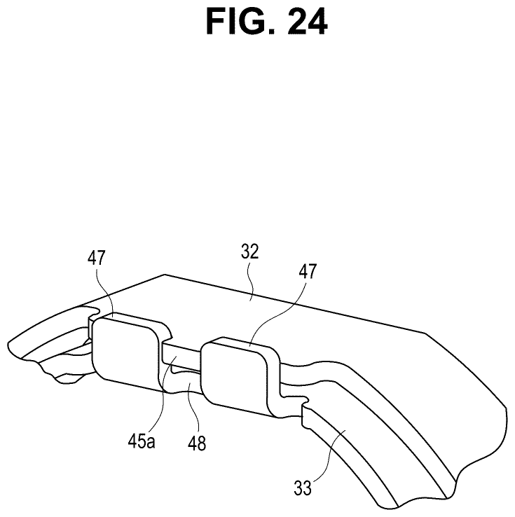

In the joining structure of the first joint ring group 30a and the second joint ring group 30b that are joint ring groups having different diameters, as shown in FIG. 23 and FIG. 24, a first joint-welding portion 45a having a protrusion shape protruded from an end portion on the proximal end side of the first linking joint ring 32 may be fitted into a groove 48 of two second joint-welding portions 47 bent from an end portion on the distal end side of the second linking joint ring 33 in the radially outward direction and welded by laser-welding LW.

The first and the second portions 47 can be spaced apart from each other in a circumferential direction of the first and the second joint ring, the end surface can be positioned between the first and second portions 47.

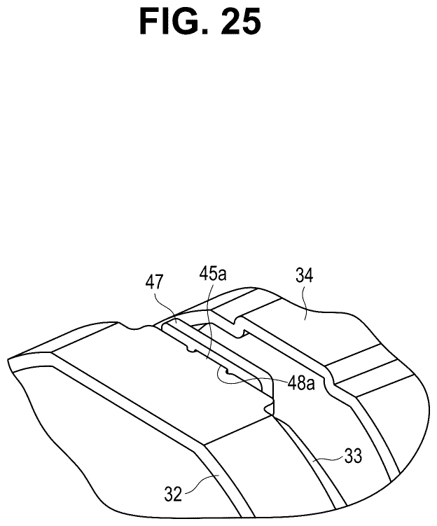

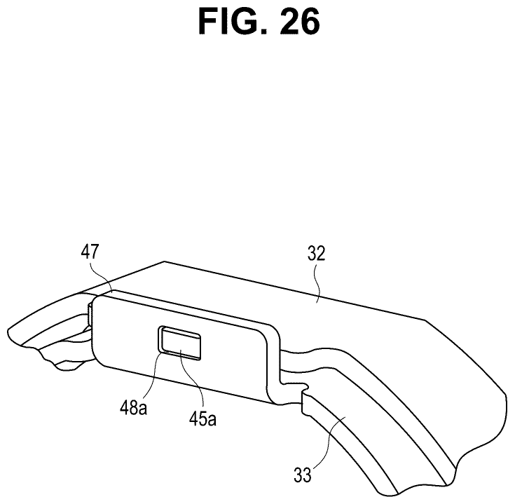

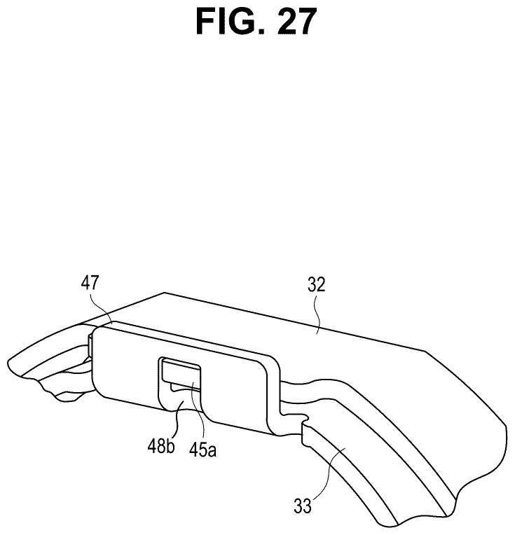

In addition, in the joining structure of the first joint ring group 30a and the second joint ring group 30b, which are joint ring groups having the different diameters, as shown in FIG. 25 to FIG. 27, the first joint-welding portion 45a may be fitted into a hole portion 48a or a long hole 48b formed in one second joint-welding portion 47 bent from the end portion on the distal end side of the second linking joint ring 33 in a radially outward direction and joined by the laser-welding LW.

Note that the first joint-welding portion 45a may be joined to the second joint-welding portion 47 by the spot-welding SW, the adhesion, or the like, other than the laser-welding LW.

### First Reference Example

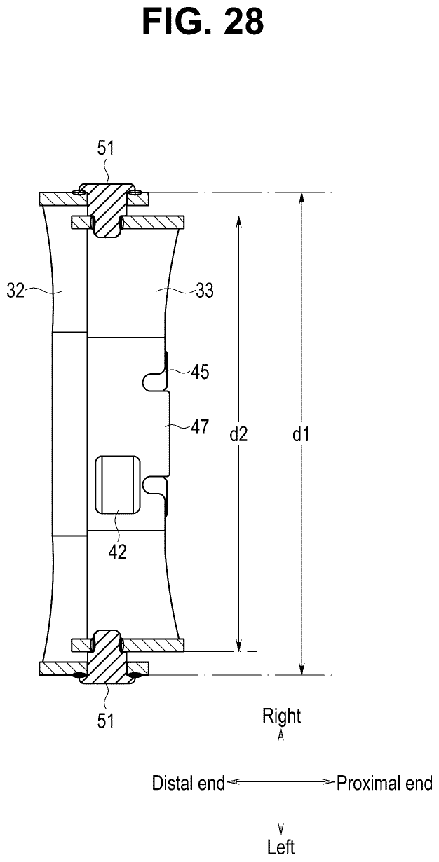

The joining method of the first linking joint ring 32 and the second linking joint ring 33 that are joint rings having different diameters may include, in addition to the laser-welding LW of the first joint-welding portion 45 and the second joint-welding portion 47, joining of the left and right directions of the first linking joint ring 32 and the second linking joint ring 33 with stepped pins 51 as shown in FIG. 28. Note that the stepped pins 51 are joined by the laser-welding LW, the spot-welding SW, the adhesion, or the like, similarly as the first joint-welding portion 45 and the second joint-welding portion 47.

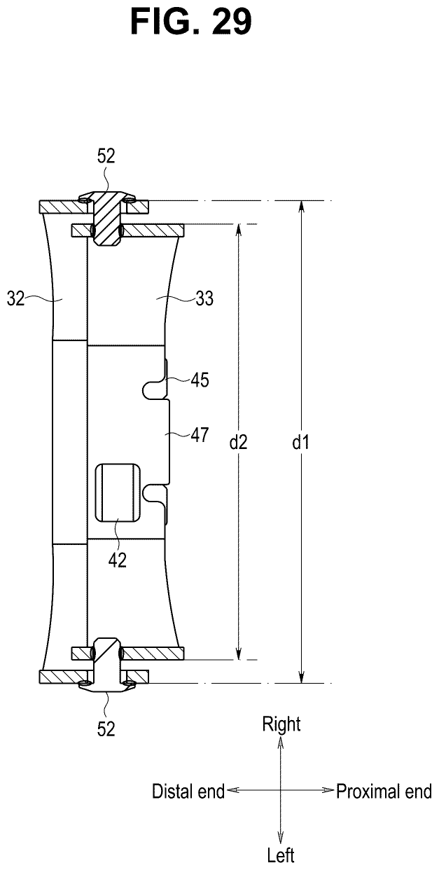

In addition, the joining method of the first linking joint ring 32 and the second linking joint ring 33 that are joint rings having different diameters may include, in addition to the laser-welding LW of the first joint-welding portion 45 and the second joint-welding portion 47, joining of the left and right directions of the first linking joint ring 32 and the second linking joint ring 33 with pins 52 as shown in FIG. 29. The pins 52 in the present example are also joined by the laser-welding LW, the spot-welding SW, adhesion, or the like, similarly as the first joint-welding portion 45 and the second joint-welding portion 47.

Thus, the stepped pins 51 or the pins 52 are added in the left and right directions of the first linking joint ring 32 and the second linking joint ring 33 that are joint rings having different diameters, to thereby be capable of reinforcing the joining strength of the first joint-welding portion 45 and the second joint-welding portion 47 by the laser-welding LW.

Thus, in the joining of the first linking joint ring 32 and the second linking joint ring 33 using the stepped pins 51 or the pins 52, approaches such as the laser-welding LW, the spot-welding SW, adhesion, and the like can be performed in the direction of the outer circumferences of the first and second linking joint rings 32, 33. This reduces the restrictions on processing, and the restrictions on the design of the inside of the bending tube 30. Furthermore, as the method of joining the first linking joint ring 32 and the second linking joint ring 33 that are joint rings having different diameters, a method of joining the first linking joint ring 32 and the second linking joint ring 33 both in a half-punched state, not shown, by welding may be employed.

### Second Reference Example

The method of joining the first linking joint ring 32 and the second linking joint ring 33 that are joint rings having different diameters may be performed without providing the first joint-welding portions 45 and the second joint-welding portions 47.

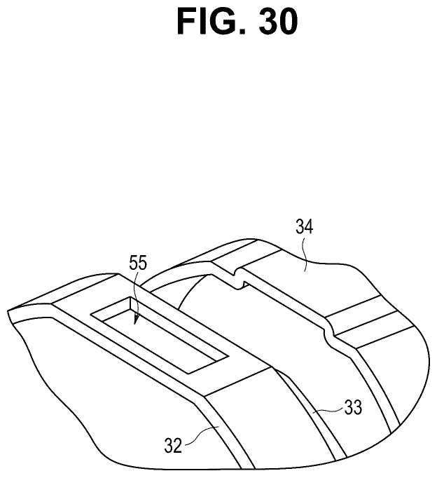

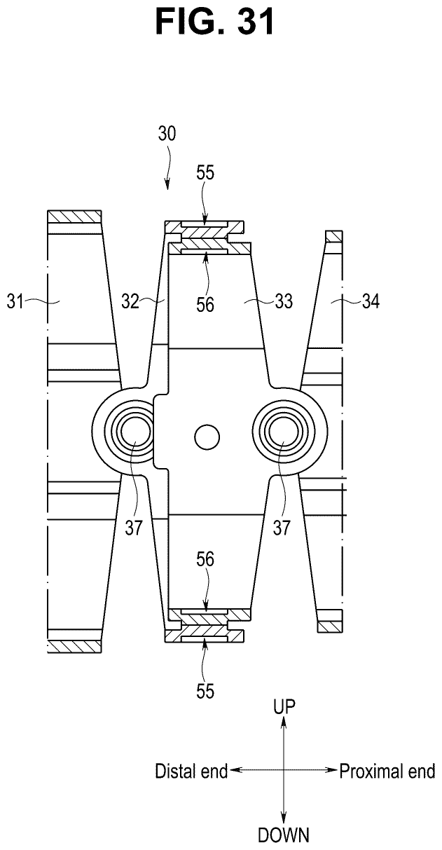

Specifically, as shown in FIG. 30 and FIG. 31, first recessed portions 55 protruding partially in the radially inward direction are formed at the upper portion and the lower portion of the first linking joint ring 32 having the large diameter, and second recessed portions 56 protruding partially in the radially outward direction are formed at the upper portion and the lower portion of the second linking joint ring 33 having the smaller diameter.

Then, the surface, which is protruded inward by each of the first recessed portions 55, of the first linking joint ring 32, and the surface, which is protruded outward by each of the second recessed portions 56, of the second linking joint ring 33 are brought into contact with each other, and the first linking joint ring 32 and the second linking joint ring 33 may be joined by the laser-welding LW, the spot-welding SW, the adhesion, or the like.

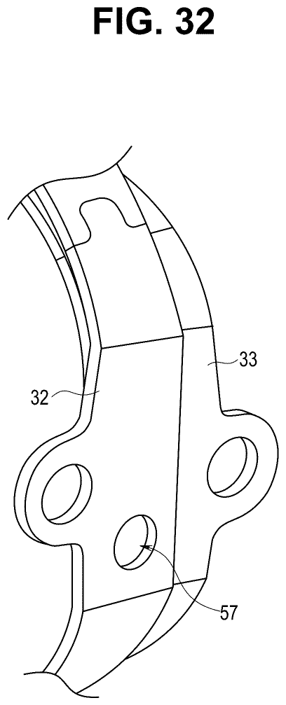

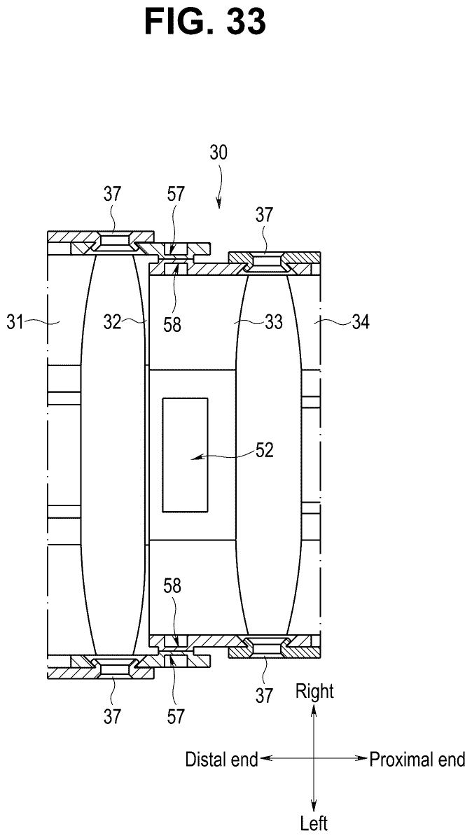

Furthermore, as shown in FIG. 32 and FIG. 33, third recessed portions 57 protruding partially in the radially inward direction are formed at the left portion and the right portion of the first linking joint ring 32 having the large diameter, and fourth recessed portions 58 protruding partially in the radially outward direction are formed at the left portion and the right portion of the second linking joint ring 33 having the smaller diameter. Then, the surfaces, which are protruded by each of the third recessed portions 57 and the fourth recessed portions 58, are brought into contact with each other, and the first linking joint ring 32 and the second linking joint ring 33 may be joined with each other by the laser-welding LW, the spot-welding SW, the adhesion, or the like.

The second reference example shows an example in which the recessed portions 55 and 56 in pairs are formed at the upper and lower positions of the first linking joint ring 32 and the second linking joint ring 33. However, depending on the difference between the outer diameter of the first linking joint ring 32 and the outer diameter of the second linking joint ring 33, only either the recessed portions 55 or the recessed portions 56 may be formed. Similarly, the second reference example shows an example in which the recessed portions 57 and 58 in pairs are formed at the left and right positions of the first linking joint ring 32 and the second linking joint ring 33. However, depending on the difference between the outer diameter of the first linking joint ring 32 and the outer diameter of the second linking joint ring 33, only either the recessed portions 57 or the recessed portions 58 may be formed. Note that each of the recessed portions 55, 56, 57, and 58 is formed by half-punching, dowel processing, and the like in the pressing processes.

### Third Reference Example

The endoscope 1 as the insertion instrument includes a wire guide 42, which is a pipe-shaped wire receiver brazed on the inner surface on the rear end side of each of the joint rings. In such a configuration, if the bending shape of the bending operation wire 44 coincides with the bending state of each of the joint rings 31, 32, 33, 34, 35, and 36, the locus of the center axis of the bending operation wire 44 does not coincide with the locus of the center axis of the wire guide 42. Thus, the bending operation wire 44 rubs, in particular, the rear end part of the wire guide 42, which results in an increase in the bending force.

If the rear end side of the wire guide 42 is processed, a difference occurs in the front side and rear side of the wire guide 42, which results in poor manufacturability. If the rear end of the wire guide 42 is made thinner to prevent the wire from being caught, for example, brazing material flows to the inner surface of the wire guide 42 when performing brazing, which results in poor manufacturability.

In addition, if the rear end of the wire guide 42 is made thinner, a gap between the bending operation wire 44 and the wire guide 42 increases partially, the force pressing the wire guide 42 by the bending operation wire 44 decreases, which causes a backlash between the joint rings 31, 32, 33, 34, 35, and 36.

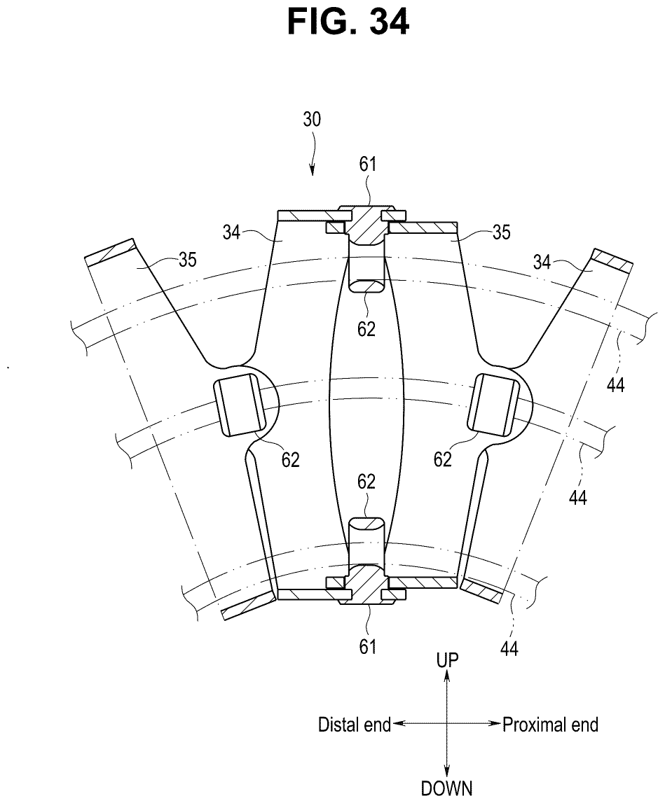

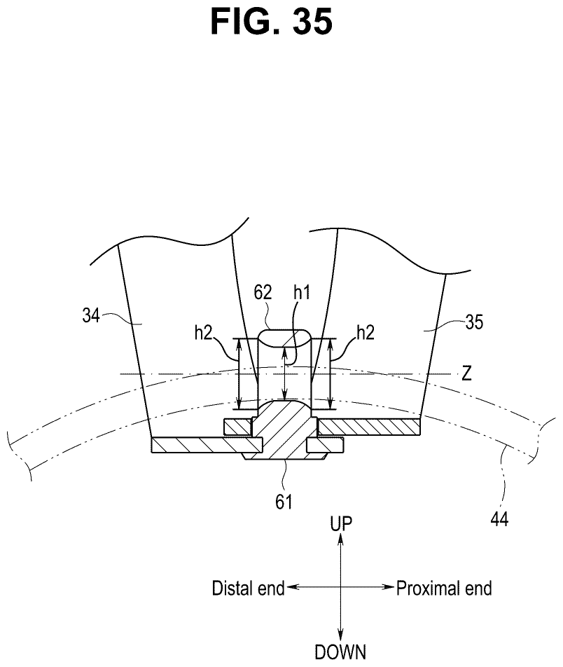

In view of the above, the bending tube 30 in the present example is configured, as shown in FIG. 34 and FIG. 35, such that a rivet 61 that rotatably couples the respective joint rings 34, 35 (31, 32, 33, 36), and the wire guide 62 which serves as the wire receiver are formed integrally.

In addition, the wire guide 62 is configured such that an inner diameter h2 of a distal end and an inner diameter h2 of a rear end of an inner surface thereof are formed to be larger than an inner diameter h1 of a center portion thereof. In other words, the wire guide 62 is formed such that the ridge line of the inner surface draws an arc in a cross section passing the center axis Z. Furthermore, this arc is set to be smaller than the radius of curvature of the bending portion 7 (˜ the radius of curvature of the bending operation wire 44).

The wire guide 62 is configured such that the inner diameters of the distal end and the rear end of the inner surface thereof are made large, to thereby be capable of preventing the rubbing of the bending operation wire 44 when the bending portion 7 is bent. In addition, the rivet 61 and the wire guide 62 are arranged at the same position of the bending tube 30 in the longitudinal direction, to allow the locus of the center axis of the wire guide 62 and the running shape of the bending operation wire 44 to be substantially coincident with each other.

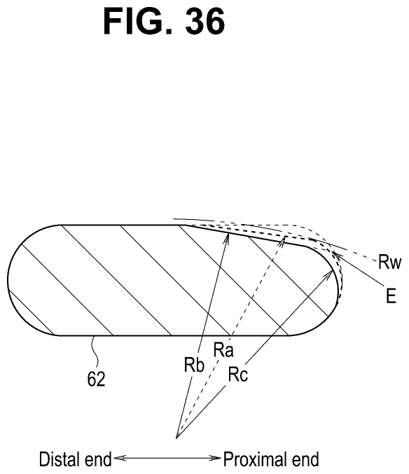

Note that, in the state where the bending portion 7 is bent at a predetermined bending angle, for example, at the maximum bending angle, the wire guide 62 may have the inner surface having the radius of curvature Rb slightly smaller than the radius of curvature Ra (Ra>Rb), as shown in FIG. 36, the radius of curvature Ra being substantially equal to the radius of curvature Rw of the bending operation wire 44 (Rw≈Ra).

Furthermore, if the wire guide 62 is formed to have the inner surface with the radius of curvature Rb, an edge E is created at the proximal end portion of the wire guide 62. Therefore, the wire guide 62 may have the inner surface having a radius of curvature Rc further smaller than Rb (Rb>Rc) such that the proximal end part is chamfered.

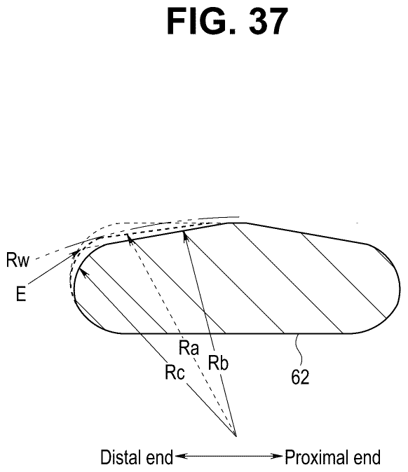

In addition, although the respective radii of curvature R for the inner surface on the proximal end side of the wire guide 62 are exemplified in FIG. 36, the inner surface on the distal end side of the wire guide 62 may also have the same configuration as that on the proximal end side, as shown in FIG. 37.

### Fourth Reference Example

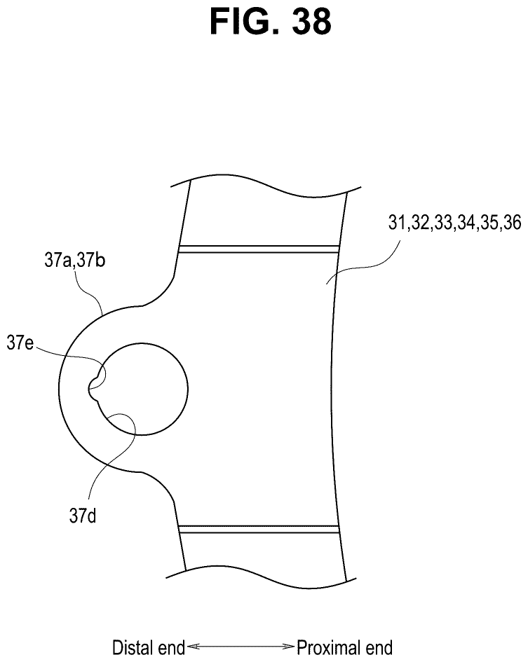

As shown in FIG. 38, each of the joint rings 31, 32, 33, 34, 35, and 36 includes a semicircular recessed portion 37e, which is a slightly deformed part, on the distal end side of the hole portion 37d of the first protrusion portion 37a or the second protrusion portion 37b. In other words, the hole portion 37d of the first protrusion portion 37a or the second protrusion portion 37b includes the recessed portion 37e which is the deformed part only in the extremely small range such that no axis shift occurs in the joint portion 37 around the distal end side. Note that the expansion of the hole area of the hole portion 37d is only slight, due to a slightly recessed shape of the recessed portion 37e.

The position where the recessed portion 37e is formed is the protruding side, i.e., the distal end side in this example, of the first protrusion portion 37a or the second protrusion portion 37b. In other words, when the bending portion 7 of the endoscope 1 is bent, a compression force is generated in a direction in which each of the joint rings 31, 32, 33, 34, 35, and 36 is pressed toward the proximal end side by a shaft member. In view of the above, the recessed portion 37e is formed on the distal end side of the hole portion 37d where no compression force is generated by the shaft member.

The disclosure recited in the above-described embodiment and the modified examples is not limited to the embodiment and the modified examples, and various modifications are possible at the practical stage in a range without departing from the gist of the disclosure. Furthermore, each of the above embodiment and modified examples includes the disclosures at various stages, and various disclosures can be extracted by appropriately combining a plurality of disclosed components.

For example, even if some of the components are removed from all the components shown in the above embodiment and modified examples, a configuration from which the components are eliminated can be extracted as a disclosure insofar as the recited problem can be solved and the recited effects of the disclosure can be obtained.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled. Example 1. A manufacturing method of a bending tube that is mounted to an insertion instrument, comprising:
forming developed shapes of a plurality of joint rings from a sheet material, the plurality of joint rings being in a state of being connected to each other by a plurality of joint portions;
forming, from the sheet material in which the developed shapes of the plurality of joint rings are formed, a plurality of first joint rings having a first diameter centered about a longitudinal axis, and a plurality of second joint rings having a second diameter different from the first diameter, by press working;
forming at least one abutting portion in at least one of the first joint rings or the second joint rings by bending a part of the sheet material in a direction intersecting the longitudinal axis, the abutting portion being configured to abut against an end surface of the other of the first joint rings or the second joint rings, the end surface being substantially orthogonal to the longitudinal axis; and
joining the at least one abutting portion formed in the one of the first joint rings or the second joint rings to the end surface of the other of the first joint rings or the second joint rings.

Example 2. The manufacturing method of the bending tube according to Example 1, further comprising,
forming the plurality of second joint rings in which the plurality of joint portions for bending the bending tube in a direction including a first directional component orthogonal to the longitudinal axis and the plurality of joint portions for bending the bending tube in a direction including a second directional component orthogonal to the longitudinal axis are alternately provided.

Example 3. The manufacturing method of the bending tube according to Example 2, wherein
the abutting portion is formed at a position shifted about the longitudinal axis, with respect to the joint portion among the joint portions of the one of the first joint rings or the second joint rings, the joint portion being closest to the other of the first joint rings or the second joint rings.

Example 4. The manufacturing method of the bending tube according to Example 3, wherein
the abutting portion is formed on a side where an angle at which the plurality of joint rings bend, with the longitudinal axis as a center, is largest.

Example 5. The manufacturing method of the bending tube according to Example 1, wherein
the abutting portion is formed by bending a protrusion portion extended from an end portion of the first joint rings or an end portion of the second joint rings in a direction away from the longitudinal axis.

Example 6. The manufacturing method of the bending tube according to Example 5, wherein
the abutting portion is set to have a dimension protruding from an outer circumference of the other of the first joint rings or the second joint rings.

Example 7. The manufacturing method of the bending tube according to Example 1, wherein
the developed shapes of the plurality of joint rings are formed in the sheet material by the press working.

Example 8. The manufacturing method of the bending tube according to Example 1, wherein
the first joint rings and the second joint rings are formed from the same sheet material, by punching processing and rounding processing by pressing.

Example 9. The manufacturing method of the bending tube according to Example 8, wherein
the first joint rings and the second joint rings are formed simultaneously from the same sheet material, by the press working.

Example 10. The manufacturing method of the bending tube according to Example 8, wherein
the abutting portion is formed simultaneously with the press working for forming the first joint rings and the second joint rings.

Example 11. The manufacturing method of the bending tube according to Example 1, wherein
the plurality of first joint rings and the plurality of second joint rings are simultaneously punched from the sheet material by pressing, and the first joint rings and the second joint rings that are adjacent to each other are coupled, to be formed by rounding processing.

Example 12. The manufacturing method of the bending tube according to Example 1, wherein
the abutting portion and a surface of an end portion in a direction of the longitudinal axis of the other of the first joint rings or the second joint rings are joined by laser-welding.

Example 13. The manufacturing method of the bending tube according to Example 12, wherein
the laser-welding is performed by applying a laser, at a predetermined angle inclined with respect to the direction of the longitudinal axis, to a contact part of the abutting portion and the surface of the end portion in the direction of the longitudinal axis of the other of the first joint rings or the second joint rings.

Example 14. The manufacturing method of the bending tube according to Example 12, wherein
the first joint rings and the second joint rings are formed from the sheet material, by punching processing and rounding processing by pressing, and
parts where base materials formed in the sheet material face each other by the rounding processing are joined to each other by the laser-welding.

Example 15. The manufacturing method of the bending tube according to Example 14, wherein
the parts where the base materials face each other by the rounding processing each include a recess and a protrusion, and a joining part where the recess and the protrusion are combined is joined by spot-welding.

Example 16. The manufacturing method of the bending tube according to Example 12, wherein
a guide member configured to guide a long member inserted through each of the joint rings is joined to an inner surface of each of the joint rings by the laser-welding.

Example 17. A bending tube that is mounted to an insertion instrument, and comprising a bending part,
the bending part comprising:
   first joint rings having a first diameter centered about a longitudinal axis, the first joint rings being formed by press-working a sheet material made of a metal plate;
   second joint rings having a second diameter centered about the longitudinal axis, the second diameter being different from the first diameter, the second joint rings being formed by press-working the sheet material;
   at least one abutting portion provided integrally with at least one of the first joint rings or the second joint rings by bending the at least one of the first joint rings or the second joint rings, and configured to abut against a surface of an end portion in a direction of the longitudinal axis of the other of the first joint rings or the second joint rings; and
   a joining portion that joins the abutting portion and the surface of the end portion in the direction of the longitudinal axis.

Example 18. The bending tube according to Example 17, wherein:
one of the first joint rings is coupled mutually with another of the first joint rings or one of the second joint rings, so that the bending tube bends in a first direction intersecting the longitudinal axis, by a pair of joint portions provided in a second direction orthogonal to the first direction;
the one of the second joint rings is coupled mutually with the other of the first joint rings or another of the second joint rings, so that the bending tube bends in the first direction, by the pair of joint portions provided in the second direction; and
the abutting portion is provided at a position shifted about the longitudinal axis, with respect to a position of one of the joint portions closest to the other of the first joint rings or the second joint rings against which the abutting portion abuts.

Example 19. An insertion instrument comprising:
an insertion portion configured to be inserted into a subject; and
a bending tube that is mounted to a distal end side of the insertion portion and includes a plurality of joint rings,
the plurality of joint rings including:
   first joint rings having a first diameter centered about a longitudinal axis, the first joint rings being formed by press-working a sheet material made of a metal plate;
   second joint rings having a second diameter centered about the longitudinal axis, the second diameter being different from the first diameter, the second joint rings being formed by press-working the sheet material;
      at least one abutting portion provided integrally with at least one of the first joint rings or the second joint rings by bending the at least one of the first joint rings or the second joint rings, and configured to abut against a surface of an end portion in a direction of the longitudinal axis of the other of the first joint rings or the second joint rings; and
      a joining portion that joins the abutting portion and the surface of the end portion in the direction of the longitudinal axis.

Example 20. The insertion instrument according to Example 19, wherein the insertion instrument is a single-use endoscope configured to be disposed of after being used once.

Example 21. A method of manufacturing a bending tube configured to be mounted to an insertion instrument, comprising:
forming a plurality of joint rings from a sheet material, wherein the plurality of joint rings are connected to each other by a plurality of joint portions, wherein the plurality of joint rings include a plurality of first joint rings having a first diameter centered about a longitudinal axis and a plurality of second joint rings centered about the longitudinal axis having a second diameter, and wherein the first diameter is different from the second diameter;
forming at least one abutting portion in at least one joint ring selected from the group consisting of the plurality of first joint rings and the plurality of second joint rings by bending a part of the sheet material in a direction intersecting the longitudinal axis, wherein the abutting portion is configured to abut against an end surface of a different joint ring selected from the group consisting of the plurality of first joint rings and the plurality of second joint rings, and wherein the end surface is substantially orthogonal to the longitudinal axis; and
joining the at least one abutting portion formed in the at least one joint ring to the end surface of the different joint ring.

Example 22. The method of manufacturing according to Example 21, wherein forming a plurality of joint rings includes forming alternately along the longitudinal axis one first joint ring of the plurality of first joint rings and one second joint ring of the plurality of second joint rings.

Example 23. The method of manufacturing according to Example 22, wherein joining the at least one abutting portion formed in the at least one joint ring to the end surface of the different joint ring forms a joint ring pair, and
wherein, for each joint ring pair and relative to the longitudinal axis, a circumferential position of the abutting portion is offset in a circumferential direction relative to a circumferential position of the joint portion.

Example 24. The method of manufacturing according to Example 23, wherein the at least one joint ring and the different joint ring forming the joint ring pair pivots relative to each other about a pivot axis at a bending angle, and
wherein the circumferential position of the abutting portion coincides with a location at which an arc length of the bending angle separating the at least one joint ring and the different joint ring forming the joint ring pair is a maximum.

Example 25. The method of manufacturing according to Example 21, wherein the part of the sheet material that is bent to form the at least one abutting portion is a protrusion portion extending from an end portion of the at least one joint ring, and
wherein forming the at least one abutting portion further includes bending the protrusion portion in a direction away from the longitudinal axis.

Example 26. The method of manufacturing according to Example 25, wherein a radially distal end of the protrusion portion is radially outward of an outer circumference of circumferential side surfaces of the joint ring pair.

Example 27. The method of manufacturing according to Example 21, wherein forming the plurality of joint rings from the sheet material is by a press working process.

Example 28. The method of manufacturing according to Example 27, wherein the pressing working process includes a punching process and a bending process.

Example 29. The method of manufacturing according to Example 28, wherein the plurality of joint rings are formed simultaneously from the sheet material.

Example 3. The method of manufacturing according to Example 28, wherein the abutting portion is formed simultaneously with the plurality of joint rings.

Example 31. The method of manufacturing according to Example 21, wherein forming the plurality of joint rings from the sheet material includes forming the plurality of first joint rings and the plurality of second joint rings simultaneously from the sheet material by a punch process, and
wherein joining the at least one abutting portion formed in the at least one joint ring to the end surface of the different joint ring, includes a bending process.

Example 32. The method of manufacturing according to Example 21, wherein joining the at least one abutting portion formed in the at least one joint ring to the end surface of the different joint ring includes joining by laser-welding.

Example 33. The method of manufacturing according to Example 32, wherein the laser-welding is performed by applying a laser, at a predetermined angle inclined with respect to a direction of the longitudinal axis, to a contact part of the abutting portion and the end surface.

Example 34. The method of manufacturing according to Example 32, wherein forming the plurality of joint rings from the sheet material includes forming the plurality of first joint rings and the plurality of second joint rings from the sheet material by a pressing working process that includes a punching process and a bending process, and
wherein portions of the plurality of joint rings that face each other by the bending process are joined to by the laser-welding.

Example 35. The method of manufacturing according to Example 34, wherein portions of the plurality of joint rings that face each other by the bending process each include a recess and a protrusion, and wherein the recess and the protrusion are joined by spot-welding.

Example 36. The method of manufacturing according to Example 32, further comprising joining a guide member to an inner surface of each of the plurality of joint rings by laser-welding, wherein the guide member is configured to guide a long member inserted through each of the plurality of joint rings.

Example 37. A bending tube that is mounted to an insertion instrument, the bending tube comprising a bending part,
wherein the bending part includes:
   a plurality of joint rings including a plurality of first joint rings and a plurality of second joint rings, wherein each first joint ring of the plurality of first joint rings has a first diameter centered about a longitudinal axis, wherein the plurality of first joint rings are formed from a sheet material made of a metal plate by a press working process, wherein each second joint ring of the plurality of second joint rings has a second diameter centered about the longitudinal axis, wherein the second diameter is different from the first diameter, and wherein the plurality of second joint rings are formed from the sheet material by the press working process;
   at least one abutting portion provided integrally with at least one joint ring selected from the group consisting of the plurality of first joint rings and the plurality of second joint rings, wherein the at least one abutting portion is a bent portion extending in a direction intersecting the longitudinal axis and wherein the abutting portion is configured to abut against an end surface of a different joint ring selected from the group consisting of the plurality of first joint rings and the plurality of second joint rings; and
   a joining portion that joins the abutting portion and the end surface in a direction of the longitudinal axis.

Example 38. The bending tube according to Example 37, wherein:
one first joint ring of the plurality of first joint rings is coupled with either (i) an other first joint ring of the plurality of first joint rings or (ii) one second joint ring of the plurality of second joint rings by a pair of joint portions,
the one second joint ring of the plurality of second joint rings is coupled with either (a) the other first joint ring of the plurality of first joint rings or (b) an other second joint ring of the plurality of second joint rings by the pair of joint portions, and
a circumferential position of the abutting portion is offset in a circumferential direction relative to a circumferential position of the pair of joint portion, and
wherein the pair of joint portions is provided in a second direction orthogonal to the first direction, and wherein the bending tube bends in a first direction intersecting the longitudinal axis.

Example 39. An insertion instrument, comprising:
an insertion portion configured to be inserted into a subject; and
a bending tube that is mounted to a distal end side of the insertion portion and that includes a plurality of joint rings,
wherein the plurality of joint rings include:
a plurality of first joint rings formed by press-working a sheet material made of a metal plate, each first joint ring of the plurality of first joint rings having a first diameter centered about a longitudinal axis,
a plurality of second joint rings formed by press-working the sheet material, each second joint ring of the plurality of second joint rings having a second diameter centered about the longitudinal axis, where the second diameter is different from the first diameter,
at least one abutting portion provided integrally with at least one joint ring selected from the group consisting of the plurality of first joint rings and the plurality of second joint rings, where the at least one abutting portion is a bent portion extending in a direction intersecting the longitudinal axis and where the abutting portion is configured to abut against an end surface of a different joint ring selected from the group consisting of the plurality of first joint rings and the plurality of second joint rings, and
a joining portion that joins the at least one abutting portion and the end surface in the direction of the longitudinal axis.

Example 40. The insertion instrument according to Example 39, wherein the insertion instrument is a single-use endoscope.

What is claimed is:

1. A method of manufacturing a bending tube for use with an insertion instrument, comprising:
forming a first joint ring from a sheet material;
forming a second joint ring from the sheet material;
forming an abutting portion in the second joint ring, wherein the abutting portion is configured to abut against an end surface of the first joint ring, and wherein the abutting portion extends in a direction intersecting a longitudinal axis; and
joining the abutting portion to the end surface of the first joint ring.

2. The method of manufacturing according to claim 1, wherein joining the abutting portion includes overlapping a proximal end portion of the first joint ring on a distal end portion of the second joint ring,
wherein the first joint ring has a first diameter centered about the longitudinal axis,
wherein the second joint ring has a second diameter centered about the longitudinal axis, and
wherein the first and the second joint rings are formed such that the first diameter is larger than the second diameter.

3. The method of manufacturing according to claim 1, wherein the first and the second joint rings forming a joint ring pair is configured to pivot relative to a joint portion of a third joint ring about a pivot axis at a bending angle, and
wherein a circumferential position of the abutting portion is offset in a circumferential direction relative to a circumferential position of the joint portion.

4. The method of manufacturing according to claim 3, wherein the circumferential position of the abutting portion coincides with a location at which an arc length of the bending angle separating the third joint ring and the joint ring pair is a maximum.

5. The method of manufacturing according to claim 1, wherein forming the abutting portion includes forming a protrusion by bending an end portion of the second joint ring in a direction away from the longitudinal axis.

6. The method of manufacturing according to claim 1, wherein forming the abutting portion to the end surface includes positioning an inner circumference of the first joint ring spaced apart from an outer circumference of the second joint ring in a radial direction of the first joint ring.

7. The method of manufacturing according to claim 1, wherein the abutting portion includes a first and a second portions spaced apart from each other in a circumferential direction of the first and the second joint ring, and
wherein joining the abutting portion to the end surface includes positioning the end surface between the first and second portions.

8. The method of manufacturing according to claim 1, wherein forming the first and the second joint rings includes a punching process and a rounding process.

9. The method of manufacturing according to claim 1, wherein joining the abutting portion to the end surface includes joining by laser-welding, and
wherein the laser-welding is performed by applying a laser, at a predetermined angle inclined with respect to the longitudinal axis, to a contact part of the abutting portion and the end surface.

10. The method of manufacturing according to claim 9, wherein the first and the second joint rings each include a recess and a protrusion, and
wherein forming the first and the second joint rings includes joining the recesses and the protrusions by spot-welding.

11. The method of manufacturing according to claim 9, further comprising joining a guide member to an inner surface of the first and the second joint rings by laser-welding,
wherein the guide member is configured to guide a wire inserted through each of the first and the second joint rings.

12. A bending tube for use with an insertion instrument, the bending tube comprising a joint ring pair,
wherein the joint ring pair includes:
a first joint ring including an end surface, and
a second joint ring including an abutting portion configured to be fixed to the end surface of the first joint ring in a longitudinal direction of the joint ring pair, wherein the abutting portion extends in a direction intersecting the longitudinal direction.

13. The bending tube according to claim 12, wherein a proximal end portion of the first joint ring is overlapped on a distal end portion of the second joint ring,
wherein the first joint ring has a first diameter centered about a longitudinal axis, the second joint ring has a second diameter centered about the longitudinal axis, and the first diameter is larger than the second diameter.

14. The bending tube according to claim 12, further comprising:
a third joint ring configured to pivot relative to the joint ring pair via a joint portion,
wherein the abutting portion includes:
a first surface configured to fixed to the end surface of the first joint ring, and
a second surface configured to contact an end surface of the third joint ring when the joint ring pair pivots relative to the third joint ring.

15. The bending tube according to claim 12, wherein a radially distal end of the abutting portion is radially outward of an outer circumference surface of the first joint ring.

16. The bending tube according to claim 12, wherein an inner circumference of the first joint ring is spaced apart from an outer circumference of the second joint ring in a radial direction of the joint ring pair.

17. The bending tube according to claim 12, wherein the abutting portion includes a first and a second portions spaced apart from each other in a circumferential direction of the first and a second portions, and
wherein joining the abutting portion to the end surface includes positioning the end surface between the first and second portions.

18. The bending tube according to claim 12, further comprising:
a third joint ring configured to pivot relative to the joint ring pair via a joint portion,
wherein a circumferential position of the abutting portion is offset in a circumferential a circumferential position of the joint portion.

19. An insertion instrument, comprising:
an insertion portion configured to be inserted into a subject; and
the bending tube according to claim 12, the bending tube mounted to a distal end side of the insertion portion and that includes a plurality of joint rings.

20. The insertion instrument according to claim 19, wherein the insertion instrument is a single-use endoscope.
